SEQUENCE LISTING (23 sequences)

SEQ ID NO: 1 - protein, 120 aa, Artificial Sequence
  Description of Artificial Sequence Synthetic polypeptide
  MISC_FEATURE (1)..(120): This sequence may encompass 1-10 “HTLQPHHHXPVV” repeating units wherein some positions may be absent
  MOD_RES (9)..(9): Leu or Ile
  MOD_RES (21)..(21): Leu or Ile
  MOD_RES (33)..(33): Leu or Ile
  MOD_RES (45)..(45): Leu or Ile
  MOD_RES (57)..(57): Leu or Ile
  MOD_RES (69)..(69): Leu or Ile
  MOD_RES (81)..(81): Leu or Ile
  MOD_RES (93)..(93): Leu or Ile
  MOD_RES (105)..(105): Leu or Ile
  MOD_RES (117)..(117): Leu or Ile
HTLQPHHHXPVVHTLQPHHHXPVVHTLQPHHHXPVVHTLQPHHHXPVVHTLQPHHHXPVVHTLQPHHHXPVVHTLQPHHHXPVVHTLQPHHHXPVVHTLQPHHHXPVVHTLQPHHHXPVV

SEQ ID NO: 2 - protein, 120 aa, Artificial Sequence
  Description of Artificial Sequence Synthetic polypeptide
  MISC_FEATURE (1)..(120): This sequence may encompass 1-10 “HTLQPHHHLPVV” repeating units wherein some positions may be absent
HTLQPHHHLPVVHTLQPHHHLPVVHTLQPHHHLPVVHTLQPHHHLPVVHTLQPHHHLPVVHTLQPHHHLPVVHTLQPHHHLPVVHTLQPHHHLPVVHTLQPHHHLPVVHTLQPHHHLPVV

SEQ ID NO: 3 - protein, 120 aa, Artificial Sequence
  Description of Artificial Sequence Synthetic polypeptide
  MISC_FEATURE (1)..(120): This sequence may encompass 1-10 “HTLQPHHHIPVV” repeating units wherein some positions may be absent
HTLQPHHHIPVVHTLQPHHHIPVVHTLQPHHHIPVVHTLQPHHHIPVVHTLQPHHHIPVVHTLQPHHHIPVVHTLQPHHHIPVVHTLQPHHHIPVVHTLQPHHHIPVVHTLQPHHHIPVV

SEQ ID NO: 4 - protein, 120 aa, Artificial Sequence
  Description of Artificial Sequence Synthetic polypeptide
  MISC_FEATURE (1)..(120): This sequence may encompass 1-10 “VPGXHSMTPXQH” repeating units wherein some positions may be absent
  MOD_RES (4)..(4): His or Gln
  MOD_RES (10)..(10): Thr or Ile
  MOD_RES (16)..(16): His or Gln
  MOD_RES (22)..(22): Thr or Ile
  MOD_RES (28)..(28): His or Gln
  MOD_RES (34)..(34): Thr or Ile
  MOD_RES (40)..(40): His or Gln
  MOD_RES (46)..(46): Thr or Ile
  MOD_RES (52)..(52): His or Gln
  MOD_RES (58)..(58): Thr or Ile
  MOD_RES (64)..(64): His or Gln
  MOD_RES (70)..(70): Thr or Ile
  MOD_RES (76)..(76): His or Gln
  MOD_RES (82)..(82): Thr or Ile
  MOD_RES (88)..(88): His or Gln
  MOD_RES (94)..(94): Thr or Ile
  MOD_RES (100)..(100): His or Gln
  MOD_RES (106)..(106): Thr or Ile
  MOD_RES (112)..(112): His or Gln
  MOD_RES (118)..(118): Thr or Ile
VPGXHSMTPXQHVPGXHSMTPXQHVPGXHSMTPXQHVPGXHSMTPXQHVPGXHSMTPXQHVPGXHSMTPXQHVPGXHSMTPXQHVPGXHSMTPXQHVPGXHSMTPXQHVPGXHSMTPXQH

SEQ ID NO: 5 - protein, 120 aa, Artificial Sequence
  Description of Artificial Sequence Synthetic polypeptide
  MISC_FEATURE (1)..(120): This sequence may encompass 1-10 “VPGHHSMTPTQH” repeating units wherein some positions may be absent
VPGHHSMTPTQHVPGHHSMTPTQHVPGHHSMTPTQHVPGHHSMTPTQHVPGHHSMTPTQHVPGHHSMTPTQHVPGHHSMTPTQHVPGHHSMTPTQHVPGHHSMTPTQHVPGHHSMTPTQH

SEQ ID NO: 6 - protein, 120 aa, Artificial Sequence
  Description of Artificial Sequence Synthetic polypeptide
  MISC_FEATURE (1)..(120): This sequence may encompass 1-10 “VPGQHSMTPIQH” repeating units wherein some positions may be absent
VPGQHSMTPIQHVPGQHSMTPIQHVPGQHSMTPIQHVPGQHSMTPIQHVPGQHSMTPIQHVPGQHSMTPIQHVPGQHSMTPIQHVPGQHSMTPIQHVPGQHSMTPIQHVPGQHSMTPIQH

SEQ ID NO: 7 - protein, 130 aa, Artificial Sequence
  Description of Artificial Sequence Synthetic polypeptide
  MISC_FEATURE (1)..(130): This sequence may encompass 1-10 “WPXTDKTKREEVD” repeating units wherein some positions may be absent
  MOD_RES (3)..(3): Ala or Ser
  MOD_RES (16)..(16): Ala or Ser
  MOD_RES (29)..(29): Ala or Ser
  MOD_RES (42)..(42): Ala or Ser
  MOD_RES (55)..(55): Ala or Ser
  MOD_RES (68)..(68): Ala or Ser
  MOD_RES (81)..(81): Ala or Ser
  MOD_RES (94)..(94): Ala or Ser
  MOD_RES (107)..(107): Ala or Ser
  MOD_RES (120)..(120): Ala or Ser
WPXTDKTKREEVDWPXTDKTKREEVDWPXTDKTKREEVDWPXTDKTKREEVDWPXTDKTKREEVDWPXTDKTKREEVDWPXTDKTKREEVDWPXTDKTKREEVDWPXTDKTKREEVDWPXTDKTKREEVD

SEQ ID NO: 8 - protein, 130 aa, Artificial Sequence
  Description of Artificial Sequence Synthetic polypeptide
  MISC_FEATURE (1)..(130): This sequence may encompass 1-10 “WPATDKTKREEVD” repeating units wherein some positions may be absent
WPATDKTKREEVDWPATDKTKREEVDWPATDKTKREEVDWPATDKTKREEVDWPATDKTKREEVDWPATDKTKREEVDWPATDKTKREEVDWPATDKTKREEVDWPATDKTKREEVDWPATDKTKREEVD

SEQ ID NO: 9 - protein, 130 aa, Artificial Sequence
  Description of Artificial Sequence Synthetic polypeptide
  MISC_FEATURE (1)..(130): This sequence may encompass 1-10 “WPSTDKTKREEVD” repeating units wherein some positions may be absent
WPSTDKTKREEVDWPSTDKTKREEVDWPSTDKTKREEVDWPSTDKTKREEVDWPSTDKTKREEVDWPSTDKTKREEVDWPSTDKTKREEVDWPSTDKTKREEVDWPSTDKTKREEVDWPSTDKTKREEVD

SEQ ID NO: 10 - protein, 160 aa, Artificial Sequence
  Description of Artificial Sequence Synthetic polypeptide
  MISC_FEATURE (1)..(160): This sequence may encompass 1-10 “HPPXHTLQPHHHXPVV” repeating units wherein some positions may be absent
  MOD_RES (4)..(4): Ser or Thr
  MOD_RES (13)..(13): Leu or Ile
  MOD_RES (20)..(20): Ser or Thr
  MOD_RES (29)..(29): Leu or Ile
  MOD_RES (36)..(36): Ser or Thr
  MOD_RES (45)..(45): Leu or Ile
  MOD_RES (52)..(52): Ser or Thr
  MOD_RES (61)..(61): Leu or Ile
  MOD_RES (68)..(68): Ser or Thr
  MOD_RES (77)..(77): Leu or Ile
  MOD_RES (84)..(84): Ser or Thr
  MOD_RES (93)..(93): Leu or Ile
  MOD_RES (100)..(100): Ser or Thr
  MOD_RES (109)..(109): Leu or Ile
  MOD_RES (116)..(116): Ser or Thr
  MOD_RES (125)..(125): Leu or Ile
  MOD_RES (132)..(132): Ser or Thr
  MOD_RES (141)..(141): Leu or Ile
  MOD_RES (148)..(148): Ser or Thr
  MOD_RES (157)..(157): Leu or Ile
HPPXHTLQPHHHXPVVHPPXHTLQPHHHXPVVHPPXHTLQPHHHXPVVHPPXHTLQPHHHXPVVHPPXHTLQPHHHXPVVHPPXHTLQPHHHXPVVHPPXHTLQPHHHXPVVHPPXHTLQPHHHXPVVHPPXHTLQPHHHXPVVHPPXHTLQPHHHXPVV

SEQ ID NO: 11 - protein, 160 aa, Artificial Sequence
  Description of Artificial Sequence Synthetic polypeptide
  MISC_FEATURE (1)..(160): This sequence may encompass 1-10 “HPPSHTLQPHHHLPVV” repeating units wherein some positions may be absent
HPPSHTLQPHHHLPVVHPPSHTLQPHHHLPVVHPPSHTLQPHHHLPVVHPPSHTLQPHHHLPVVHPPSHTLQPHHHLPVVHPPSHTLQPHHHLPVVHPPSHTLQPHHHLPVVHPPSHTLQPHHHLPVVHPPSHTLQPHHHLPVVHPPSHTLQPHHHLPVV

SEQ ID NO: 12 - protein, 160 aa, Artificial Sequence
  Description of Artificial Sequence Synthetic polypeptide
  MISC_FEATURE (1)..(160): This sequence may encompass 1-10 “HPPTHTLQPHHHIPVV” repeating units wherein some positions may be absent
HPPTHTLQPHHHIPVVHPPTHTLQPHHHIPVVHPPTHTLQPHHHIPVVHPPTHTLQPHHHIPVVHPPTHTLQPHHHIPVVHPPTHTLQPHHHIPVVHPPTHTLQPHHHIPVVHPPTHTLQPHHHIPVVHPPTHTLQPHHHIPVVHPPTHTLQPHHHIPVV

SEQ ID NO: 13 - protein, 220 aa, Artificial Sequence
  Description of Artificial Sequence Synthetic polypeptide
  MISC_FEATURE (1)..(220): This sequence may encompass 1-10 “PGYINXSYEXSHSQAINXDRTA” repeating units wherein some positions may be absent
  MOD_RES (6)..(6): Leu or Phe
  MOD_RES (10)..(10): Lys or Asn
  MOD_RES (18)..(18): Thr or Val
  MOD_RES (28)..(28): Leu or Phe
  MOD_RES (32)..(32): Lys or Asn
  MOD_RES (40)..(40): Thr or Val
  MOD_RES (50)..(50): Leu or Phe
  MOD_RES (54)..(54): Lys or Asn
  MOD_RES (62)..(62): Thr or Val
  MOD_RES (72)..(72): Leu or Phe
  MOD_RES (76)..(76): Lys or Asn
  MOD_RES (84)..(84): Thr or Val
  MOD_RES (94)..(94): Leu or Phe
  MOD_RES (98)..(98): Lys or Asn
  MOD_RES (106)..(106): Thr or Val
  MOD_RES (116)..(116): Leu or Phe
  MOD_RES (120)..(120): Lys or Asn
  MOD_RES (128)..(128): Thr or Val
  MOD_RES (138)..(138): Leu or Phe
  MOD_RES (142)..(142): Lys or Asn
  MOD_RES (150)..(150): Thr or Val
  MOD_RES (160)..(160): Leu or Phe
  MOD_RES (164)..(164): Lys or Asn
  MOD_RES (172)..(172): Thr or Val
  MOD_RES (182)..(182): Leu or Phe
  MOD_RES (186)..(186): Lys or Asn
  MOD_RES (194)..(194): Thr or Val
  MOD_RES (204)..(204): Leu or Phe
  MOD_RES (208)..(208): Lys or Asn
  MOD_RES (216)..(216): Thr or Val
PGYINXSYEXSHSQAINXDRTAPGYINXSYEXSHSQAINXDRTAPGYINXSYEXSHSQAINXDRTAPGYINXSYEXSHSQAINXDRTAPGYINXSYEXSHSQAINXDRTAPGYINXSYEXSHSQAINXDRTAPGYINXSYEXSHSQAINXDRTAPGYINXSYEXSHSQAINXDRTAPGYINXSYEXSHSQAINXDRTAPGYINXSYEXSHSQAINXDRTA

SEQ ID NO: 14 - protein, 220 aa, Artificial Sequence
  Description of Artificial Sequence Synthetic polypeptide
  MISC_FEATURE (1)..(220): This sequence may encompass 1-10 “PGYINLSYEKSHSQAINTDRTA” repeating units wherein some positions may be absent
PGYINLSYEKSHSQAINTDRTAPGYINLSYEKSHSQAINTDRTAPGYINLSYEKSHSQAINTDRTAPGYINLSYEKSHSQAINTDRTAPGYINLSYEKSHSQAINTDRTAPGYINLSYEKSHSQAINTDRTAPGYINLSYEKSHSQAINTDRTAPGYINLSYEKSHSQAINTDRTAPGYINLSYEKSHSQAINTDRTAPGYINLSYEKSHSQAINTDRTA

SEQ ID NO: 15 - protein, 220 aa, Artificial Sequence
  Description of Artificial Sequence Synthetic polypeptide
  MISC_FEATURE (1)..(220): This sequence may encompass 1-10 “PGYINFSYENSHSQAINVDRTA” repeating units wherein some positions may be absent
PGYINFSYENSHSQAINVDRTAPGYINFSYENSHSQAINVDRTAPGYINFSYENSHSQAINVDRTAPGYINFSYENSHSQAINVDRTAPGYINFSYENSHSQAINVDRTAPGYINFSYENSHSQAINVDRTAPGYINFSYENSHSQAINVDRTAPGYINFSYENSHSQAINVDRTAPGYINFSYENSHSQAINVDRTAPGYINFSYENSHSQAINVDRTA

SEQ ID NO: 16 - protein, 15 aa, Artificial Sequence
  Description of Artificial Sequence Synthetic peptide
SYENSHSQAINVDRT

SEQ ID NO: 17 - protein, 240 aa, Artificial Sequence
  Description of Artificial Sequence Synthetic polypeptide
  MISC_FEATURE (1)..(240): This sequence may encompass 1-10 “LPPLFSMPLSPILPELPLEAWPAT” repeating units wherein some positions may be absent
LPPLFSMPLSPILPELPLEAWPATLPPLFSMPLSPILPELPLEAWPATLPPLFSMPLSPILPELPLEAWPATLPPLFSMPLSPILPELPLEAWPATLPPLFSMPLSPILPELPLEAWPATLPPLFSMPLSPILPELPLEAWPATLPPLFSMPLSPILPELPLEAWPATLPPLFSMPLSPILPELPLEAWPATLPPLFSMPLSPILPELPLEAWPATLPPLFSMPLSPILPELPLEAWPAT

SEQ ID NO: 18 - protein, 420 aa, Artificial Sequence
  Description of Artificial Sequence Synthetic polypeptide
  MISC_FEATURE (1)..(420): This sequence may encompass 1-10 “HPPXHTLQPHHHXPVVPAQQPVXPQQPMMPVPGXHSMTPXQH” repeating units wherein some positions may be absent
  MOD_RES (4)..(4): Ser or Thr
  MOD_RES (13)..(13): Leu or Ile
  MOD_RES (23)..(23): Ala or Ile
  MOD_RES (34)..(34): His or Gln
  MOD_RES (40)..(40): Thr or Ile
  MOD_RES (46)..(46): Ser or Thr
  MOD_RES (55)..(55): Leu or Ile
  MOD_RES (65)..(65): Ala or Ile
  MOD_RES (76)..(76): His or Gln
  MOD_RES (82)..(82): Thr or Ile
  MOD_RES (88)..(88): Ser or Thr
  MOD_RES (97)..(97): Leu or Ile
  MOD_RES (107)..(107): Ala or Ile
  MOD_RES (118)..(118): His or Gln
  MOD_RES (124)..(124): Thr or Ile
  MOD_RES (130)..(130): Ser or Thr
  MOD_RES (139)..(139): Leu or Ile
  MOD_RES (149)..(149): Ala or Ile
  MOD_RES (160)..(160): His or Gln
  MOD_RES (166)..(166): Thr or Ile
  MOD_RES (172)..(172): Ser or Thr
  MOD_RES (181)..(181): Leu or Ile
  MOD_RES (191)..(191): Ala or Ile
  MOD_RES (202)..(202): His or Gln
  MOD_RES (208)..(208): Thr or Ile
  MOD_RES (214)..(214): Ser or Thr
  MOD_RES (223)..(223): Leu or Ile
  MOD_RES (233)..(233): Ala or Ile
  MOD_RES (244)..(244): His or Gln
  MOD_RES (250)..(250): Thr or Ile
  MOD_RES (256)..(256): Ser or Thr
  MOD_RES (265)..(265): Leu or Ile
  MOD_RES (275)..(275): Ala or Ile
  MOD_RES (286)..(286): His or Gln
  MOD_RES (292)..(292): Thr or Ile
  MOD_RES (298)..(298): Ser or Thr
  MOD_RES (307)..(307): Leu or Ile
  MOD_RES (317)..(317): Ala or Ile
  MOD_RES (328)..(328): His or Gln
  MOD_RES (334)..(334): Thr or Ile
  MOD_RES (340)..(340): Ser or Thr
  MOD_RES (349)..(349): Leu or Ile
  MOD_RES (359)..(359): Ala or Ile
  MOD_RES (370)..(370): His or Gln
  MOD_RES (376)..(376): Thr or Ile
  MOD_RES (382)..(382): Ser or Thr
  MOD_RES (391)..(391): Leu or Ile
  MOD_RES (401)..(401): Ala or Ile
  MOD_RES (412)..(412): His or Gln
  MOD_RES (418)..(418): Thr or Ile
HPPXHTLQPHHHXPVVPAQQPVXPQQPMMPVPGXHSMTPXQHHPPXHTLQPHHHXPVVPAQQPVXPQQPMMPVPGXHSMTPXQHHPPXHTLQPHHHXPVVPAQQPVXPQQPMMPVPGXHSMTPXQHHPPXHTLQPHHHXPVVPAQQPVXPQQPMMPVPGXHSMTPXQHHPPXHTLQPHHHXPVVPAQQPVXPQQPMMPVPGXHSMTPXQHHPPXHTLQPHHHXPVVPAQQPVXPQQPMMPVPGXHSMTPXQHHPPXHTLQPHHHXPVVPAQQPVXPQQPMMPVPGXHSMTPXQHHPPXHTLQPHHHXPVVPAQQPVXPQQPMMPVPGXHSMTPXQHHPPXHTLQPHHHXPVVPAQQPVXPQQPMMPVPGXHSMTPXQHHPPXHTLQPHHHXPVVPAQQPVXPQQPMMPVPGXHSMTPXQH

SEQ ID NO: 19 - protein, 420 aa, Artificial Sequence
  Description of Artificial Sequence Synthetic polypeptide
  MISC_FEATURE (1)..(420): This sequence may encompass 1-10 “HPPSHTLQPHHHLPVVPAQQPVAPQQPMMPVPGHHSMTPTQH” repeating units wherein some positions may be absent
HPPSHTLQPHHHLPVVPAQQPVAPQQPMMPVPGHHSMTPTQHHPPSHTLQPHHHLPVVPAQQPVAPQQPMMPVPGHHSMTPTQHHPPSHTLQPHHHLPVVPAQQPVAPQQPMMPVPGHHSMTPTQHHPPSHTLQPHHHLPVVPAQQPVAPQQPMMPVPGHHSMTPTQHHPPSHTLQPHHHLPVVPAQQPVAPQQPMMPVPGHHSMTPTQHHPPSHTLQPHHHLPVVPAQQPVAPQQPMMPVPGHHSMTPTQHHPPSHTLQPHHHLPVVPAQQPVAPQQPMMPVPGHHSMTPTQHHPPSHTLQPHHHLPVVPAQQPVAPQQPMMPVPGHHSMTPTQHHPPSHTLQPHHHLPVVPAQQPVAPQQPMMPVPGHHSMTPTQHHPPSHTLQPHHHLPVVPAQQPVAPQQPMMPVPGHHSMTPTQH

SEQ ID NO: 20 - protein, 420 aa, Artificial Sequence
  Description of Artificial Sequence Synthetic polypeptide
  MISC_FEATURE (1)..(420): This sequence may encompass 1-10 “HPPTHTLQPHHHIPVVPAQQPVIPQQPMMPVPGQHSMTPIQH” repeating units wherein some positions may be absent
HPPTHTLQPHHHIPVVPAQQPVIPQQPMMPVPGQHSMTPIQHHPPTHTLQPHHHIPVVPAQQPVIPQQPMMPVPGQHSMTPIQHHPPTHTLQPHHHIPVVPAQQPVIPQQPMMPVPGQHSMTPIQHHPPTHTLQPHHHIPVVPAQQPVIPQQPMMPVPGQHSMTPIQHHPPTHTLQPHHHIPVVPAQQPVIPQQPMMPVPGQHSMTPIQHHPPTHTLQPHHHIPVVPAQQPVIPQQPMMPVPGQHSMTPIQHHPPTHTLQPHHHIPVVPAQQPVIPQQPMMPVPGQHSMTPIQHHPPTHTLQPHHHIPVVPAQQPVIPQQPMMPVPGQHSMTPIQHHPPTHTLQPHHHIPVVPAQQPVIPQQPMMPVPGQHSMTPIQHHPPTHTLQPHHHIPVVPAQQPVIPQQPMMPVPGQHSMTPIQH

SEQ ID NO: 21 - protein, 140 aa, Artificial Sequence
  Description of Artificial Sequence Synthetic polypeptide
  MISC_FEATURE (1)..(140): This sequence may encompass 1-10 “PAQQPVXPQQPMMP” repeating units wherein some positions may be absent
  MOD_RES (7)..(7): Ala or Ile
  MOD_RES (21)..(21): Ala or Ile
  MOD_RES (35)..(35): Ala or Ile
  MOD_RES (49)..(49): Ala or Ile
  MOD_RES (63)..(63): Ala or Ile
  MOD_RES (77)..(77): Ala or Ile
  MOD_RES (91)..(91): Ala or Ile
  MOD_RES (105)..(105): Ala or Ile
  MOD_RES (119)..(119): Ala or Ile
  MOD_RES (133)..(133): Ala or Ile
PAQQPVXPQQPMMPPAQQPVXPQQPMMPPAQQPVXPQQPMMPPAQQPVXPQQPMMPPAQQPVXPQQPMMPPAQQPVXPQQPMMPPAQQPVXPQQPMMPPAQQPVXPQQPMMPPAQQPVXPQQPMMPPAQQPVXPQQPMMP

SEQ ID NO: 22 - protein, 140 aa, Artificial Sequence
  Description of Artificial Sequence Synthetic polypeptide
  MISC_FEATURE (1)..(140): This sequence may encompass 1-10 “PAQQPVAPQQPMMP” repeating units wherein some positions may be absent
PAQQPVAPQQPMMPPAQQPVAPQQPMMPPAQQPVAPQQPMMPPAQQPVAPQQPMMPPAQQPVAPQQPMMPPAQQPVAPQQPMMPPAQQPVAPQQPMMPPAQQPVAPQQPMMPPAQQPVAPQQPMMPPAQQPVAPQQPMMP

SEQ ID NO: 23 - protein, 140 aa, Artificial Sequence
  Description of Artificial Sequence Synthetic polypeptide
  MISC_FEATURE (1)..(140): This sequence may encompass 1-10 “PAQQPVIPQQPMMP” repeating units wherein some positions may be absent
PAQQPVIPQQPMMPPAQQPVIPQQPMMPPAQQPVIPQQPMMPPAQQPVIPQQPMMPPAQQPVIPQQPMMPPAQQPVIPQQPMMPPAQQPVIPQQPMMPPAQQPVIPQQPMMPPAQQPVIPQQPMMPPAQQPVIPQQPMMP

(19) United States
(12) Patent Application Publication
(10) Pub. No.: US 2018/0200172 A1
(43) Pub. Date: Jul. 19, 2018

(54) REAGENTS AND METHODS FOR WHITENING TEETH

(71) Applicant: UNIVERSITY OF WASHINGTON THROUGH ITS CENTER FOR COMMERCIALIZATION, Seattle, WA (US)

(72) Inventors: Mehmet Sarikaya, Seattle, WA (US); Hanson Kwok Fong, Bothell, WA (US); Deniz Tanil Yucesoy, Seattle, WA (US)

(73) Assignee: UNIVERSITY OF WASHINGTON THROUGH ITS CENTER FOR COMMERCIALIZATION, Seattle, WA (US)

(21) Appl. No.: 15/542,902

(22) PCT Filed: Jan. 13, 2016
(86) PCT No.: PCT/US16/13301

§ 371 Date: Jul. 11, 2017

Related U.S. Application Data
(60) Provisional application No. 62/102,855, filed on Jan. 13, 2015.

(51) Int. Cl. A61K 8/64; A61Q 11/02

(52) U.S. Cl. CPC A61K 8/64; A61Q 11/02

(57) ABSTRACT

Reagents and methods for whitening teeth using biomineralizing peptides are described.

### CROSS-REFERENCE TO RELATED APPLICATIONS

This application claims priority from U.S. Provisional Patent Application Ser. No. 62/102,855, filed 13 Jan. 2015, entitled “REAGENTS AND METHODS FOR WHITENING TEETH”, the contents of which are incorporated herein by reference.

### SEQUENCE LISTING

The instant application contains a Sequence Listing which has been submitted electronically in ASCII format and is hereby incorporated by reference in its entirety. Said ASCII copy, created on Jan. 12, 2015, is named 091619-1036_SL.txt and is 52,347 bytes in size.

### BACKGROUND

Accumulation of various chromogens/discolorants, for example, food and tobacco that come into contact daily onto tooth surfaces and their subsequent penetration into deeper regions (dentin), cause tooth discoloration. Furthermore, the process of aging, disease, trauma, certain medications, certain congenital conditions, and environmental effects can also cause teeth to become discolored. Although discolored teeth do not cause health problems, since bright white teeth are usually considered to be cosmetically desirable, there is a great deal of interest in developing compositions and methods for whitening teeth.

There are several techniques for whitening or bleaching of teeth. Professional whitening methods, also known as “in-clinic” whitening strategies, are considered presently as the most effective methods. These in-clinic whitening strategies typically involve the application of high-concentration peroxide products (up to 35%) and other abrasive chemicals to the discolored area. These peroxide species penetrate the stained area (up to underlying dentin layer) and dissolve the accumulated discoloring agents through an oxidation process. To achieve desired effects more quickly, such oxidation reactions on teeth are often assisted with external laser light application, which heats up the reaction site and thereby increases the rate oxidation reaction. Furthermore, utilization of titanium dioxide up to 10% is often preferred to facilitate these photo-catalytic reactions. Other in-clinic whitening techniques involve superficially removing the enamel layer with abrasive instruments or pumice followed by treatment with additional caustic agents.

There are several drawbacks of these in-clinic techniques. First of all, the abrasive chemicals and peroxide agents causes enamel demineralization and results with teeth sensitivity. In most cases sore/injured gums as well as bad taste of the product itself cause much discomfort to the patients. Further, patients are required to make a clinic appointment to get this medical service.

Other existing products for at-home use contain considerably lower concentrations of active oxidizing agents and, thus, are generally less effective than in-clinic whitening strategies and products. Therefore, dramatic whitening effect can only be achieved by the repeated applications of these reagents for several weeks. These treatments often assisted with bleaching trays (night-guard) in order to better localize bleaching products and, thereby, maximize the whitening effect. However, even though these at home products contain active whitening agents in lower concentrations, similar side effects as those associated with professional treatments are very common. In addition, there are paint-on, at-home whitening products, also known as “tooth varnishes”, as well as whitening strips intended to eliminate the need for dental trays. However, these products require more frequent applications, usually 3-times in a day, to complete whitening procedure. Finally, among the variety of at-home use products, whitening toothpastes and gels are the least effective form of whitening products due to their short contact time with the tooth surfaces. Although bleaching agent additives augment the effectiveness, the whitening effect is primarily as a result of removal of surface stains via mechanical action of brushing and other polishing ingredients (for example, silica particles).

There is presently an unmet need for tooth whitening methods and products that reduce or eliminate the need for concentrated, abrasive oxidizing agents and attendant side effects such as demineralization-associated tooth sensitivity and gum line injuries.

### SUMMARY

Herein we provide methods and compositions for whitening teeth where the natural color of teeth is restored and improved upon by generating newly formed thin mineral layer on discolored tooth surface using one or more biomineralizing polypeptides.

In a first aspect, the present application provides a method for whitening teeth, comprising administering to a subject in need thereof an amount effective to whiten the teeth of a biomineralizing polypeptide. In some embodiments, the method for whitening teeth comprises administering to a subject in need thereof an effective amount of a pharmaceutical composition comprising a biomineralizing polypeptide. In some embodiments, the biomineralizing polypeptide comprises an amino acid sequence selected from the group consisting of:

(ADP3; SEQ ID NO: 7)
(WP(A/S)TDKTKREEVD)₁₋₁₀;
(ADP5; SEQ ID NO: 13)
(PGYTN(L/F)SYE(K/N)SHSQAIN(T/V)DRTA)₁₋₁₀;
(ADP6; SEQ ID NO: 17)
(LPPLFSMPLSPILPELPLEAWPAT)₁₋₁₀;
(ADP7; SEQ ID NO: 18)
(HPP(S/T)HTLQPHHH(L/I)PVVPAQQPV(A/I)PQQPMMPVPG
(H/Q)HSMTP(T/I)QH)₁₋₁₀;
and
(ADP7; SEQ ID NO: 18)
12-42 contiguous amino acids of (HPP(S/T)HTLQPHHH
(L/I)PVVPAQQPV(A/I)PQQPMMPVPG(H/Q)HSMTP(T/I)
QH)₁₋₁₀;

or a functional equivalent thereof, or any combination thereof. In some embodiments, the biomineralizing polypeptide comprises the amino acid sequence (PGYIN(L/F)SYE(K/N) SHSQAIN(T/V)DRTA)₁₋₁₀ (ADP5; SEQ ID NO:13), or a functional equivalent thereof. In some embodiments, the biomineralizing polypeptide comprises the amino acid sequence (PGYINFSYENSHSQAINVDRTA)₁₋₁₀ (ADP5H; SEQ ID NO:15), or a functional equivalent thereof. In some embodiments, the biomineralizing polypeptide comprises the amino acid sequence (SYENSHSQAINVDRT)₁₋₁₀ (shADP5; SEQ ID NO:16), or a functional equivalent thereof. In some embodiments, the biomineralizing polypeptide comprises (HPP(S/T)HTLQPHHH(L/I)PVVPAQQPV(A/I)PQ QPMMPVPG(H/Q)HSMTP(T/I)QH)₁₋₁₀ (ADP7; SEQ ID NO:18), 12-42 contiguous amino acids of (HPP(S/T)HTLQPHHH(L/I) PVVPAQQPV(A/I)PQQPMMPVPG(H/Q)HSMTP(T/I)QH)₁₋₁₀ (ADP7; SEQ ID NO:18), or a functional equivalent thereof. In some embodiments, the biomineralizing polypeptide comprises an amino acid sequence selected from the group consisting of:

(ADP1)
(SEQ ID NO: 1)
(HTLQPHHH(L/I)PVV)₁₋₁₀;
(ADP2)
(SEQ ID NO: 4)
(VPG(H/Q)HSMTP(T/I)QH)₁₋₁₀
(ADP4)
(SEQ ID NO: 10)
(HPP(S/T)HTLQPHHH(L/I)PVV)₁₋₁₀;
(ADP8)
(SEQ ID NO: 21)
(PAQQPV(A/I)PQQPMMP)₁₋₁₀;
(ADP7)
(SEQ ID NO: 18)
(HPP(S/T)HTLQPHHH(L/I)PVVPAQQPV(A/I)PQQPMMPVP(H/Q)
HSMTP(T/I)QH)₁₋₁₀;
(ADP1M)
(SEQ ID NO: 2)
(HTLQPHHHLPVV)₁₋₁₀;
(ADP1H)
(SEQ ID NO: 3)
(HTLQPHHHIPVV)₁₋₁₀;
(ADP2M)
(SEQ ID NO: 5)
(VPGHHSMTPTQH)₁₋₁₀;
(ADP2H)
(SEQ ID NO: 6)
(VPGQHSMTPIQH)₁₋₁₀;
(ADP4M)
(SEQ ID NO: 11)
(HPPSHTLQPHHELPVV)₁₋₁₀;
(ADP4H)
(SEQ ID NO: 12)
(HPPTHTLQPHHHIPVV)₁₋₁₀;
(ADP7M)
(SEQ ID NO: 19)
(HPPSHTLQPHHELPVVPAQQPVAPQQPMMPVPGHHSMTPTQH)₁₋₁₀;
(ADP7H)
(SEQ ID NO: 20)
(HPPTHTLQPHHHIPVVPAQQPVIPQQPMMPVPGQHSMTPIQH)₁₋₁₀;
(ADP8M)
(SEQ ID NO: 22)
(PAQQPVAPQQPMMP)₁₋₁₀;
and
(ADP8H)
(SEQ ID NO: 23)
(PAQQPVIPQQPMMP)₁₋₁₀;

or a functional equivalent thereof, or any combination thereof. In some embodiments, the biomineralizing polypeptide comprises an amino acid sequence selected from the group consisting of:

(ADP3M)
(SEQ ID NO: 8)
(WPATDKTKREEVD)₁₋₁₀;
and
(ADP3H)
(SEQ ID NO: 9)
(WPSTDKTKREEVD)₁₋₁₀,

or a functional equivalent thereof, or a combination thereof. In some embodiments, the biomineralizing polypeptide comprises one or more fusion peptides, wherein each of the one or more fusion peptides independently comprises two or more amino acid sequences selected from the group consisting of:

(ADP1)
(SEQ ID NO: 1)
(HTLQPHHH(L/I)PVV)₁₋₁₀;
(ADP2)
(SEQ ID NO: 4)
(VPG(H/Q)HSMTP(T/I)QH)₁₋₁₀
(ADP3)
(SEQ ID NO: 7)
WP(A/S)TDKTKREEVD₁₋₁₀;
(ADP4)
(SEQ ID NO: 10)
(HPP(S/T)HTLQPHHH(L/I)PVV)₁₋₁₀;
(ADP5)
(SEQ ID NO: 13)
(PGYIN(L/F)SYE(K/N)SHSQAIN(T/V)DRTA)₁₋₁₀;
(ADP6)
(SEQ ID NO: 17)
(LPPLFSMPLSPILPELPLEAWPAT)₁₋₁₀;
(ADP8)
(SEQ ID NO: 21)
(PAQQPV(A/I)PQQPMMP)₁₋₁₀;
(ADP7)
(SEQ ID NO: 18)
(HPP(S/T)HTLQPHHH(L/I)PVVPAQQPV(A/I)PQQPMMPVP(H/Q)
HSMTP(T/I)QH)₁₋₁₀;
(ADP1M)
(SEQ ID NO: 2)
(HTLQPHHHLPVV)₁₋₁₀;
(ADP1H)
(SEQ ID NO: 3)
(HTLQPHHHIPVV)₁₋₁₀;
(ADP2M)
(SEQ ID NO: 5)
(VPGHHSMTPTQH)₁₋₁₀;
(ADP2H)
(SEQ ID NO: 6)
(VPGQHSMTPIQH)₁₋₁₀;
(ADP4M)
(SEQ ID NO: 11)
(HPPSHTLQPHHELPVV)₁₋₁₀;
(ADP4H)
(SEQ ID NO: 12)
(HPPTHTLQPHHHIPVV)₁₋₁₀;
(ADP7M)
(SEQ ID NO: 19)
(HPPSHTLQPHHELPVVPAQQPVAPQQPMMPVPGHHSMTPTQH)₁₋₁₀;
(ADP7H)
(SEQ ID NO: 20)
(HPPTHTLQPHHHIPVVPAQQPVIPQQPMMPVPGQHSMTPIQH)₁₋₁₀;
(ADP8M)
(SEQ ID NO: 22)
(PAQQPVAPQQPMMP)₁₋₁₀;
and
(ADP8H)
(SEQ ID NO: 23)
(PAQQPVIPQQPMMP)₁₋₁₀;

or a functional equivalent thereof. In some embodiments, the biomineralizing polypeptide further comprises a fluorescent agent. In some embodiments, the method further comprises applying light to the teeth of the subject, thereby further whitening the teeth. In some embodiments, the light is selected from one of a diode laser; a PAC light; and a halogen light. In some embodiments, the method further comprises administering at least one cleaning agent to the teeth of the subject. In some embodiments, the method further comprises administering hydrogen peroxide, carbamide peroxide, titanium dioxide, nano-hydroxyapatite particles, zirconia powder, or any combination thereof to the teeth of the subject.

In a second aspect, the present application provides an oral care product, comprising at least one biomineralizing polypeptide, at least one calcium ion source, and at least one phosphate ion source. The oral care product can further comprise at least one cleaning agent. In some embodiments, the at least one cleaning agent is selected from the group consisting of hydrogen peroxide, titanium dioxide, carbamide peroxide, nano-hydroxyapatite particles, zirconia powder, or any combination thereof. The calcium ion source is generally any calcium salt. In some embodiments, the calcium ion source is selected from the group consisting of calcium acetate, calcium carbonate, calcium citrate, calcium chloride, calcium gluconate, calcium glycerophosphate, calcium lactate, and calcium phosphate. The phosphate ion source is generally any phosphate salt. In some embodiments, the phosphate ion source is selected from the group consisting of aluminum phosphates, calcium phosphates, potassium phosphates, and sodium phosphates. In some embodiments, the oral care product is selected from the group consisting of toothpaste, toothpowders, mouthwash, gel, dental floss, liquid dentifrices, dental tablets, topical gels, troches, chewing gums, dental pastes, gingival massage creams, gargle tablets, lozenges, tooth trays, tooth varnishes, and food products. In some embodiments, the biomineralizing polypeptide comprises an amino acid sequence selected from the group consisting of:

(ADP3; SEQ ID NO: 7)
(WP(A/S)TDKTKREEVD)₁₋₁₀;
(ADP5; SEQ ID NO: 13)
(PGYTN(L/F)SYE(K/N)SHSQAIN(T/V)DRTA)₁₋₁₀;
(ADP6; SEQ ID NO: 17)
(LPPLFSMPLSPILPELPLEAWPAT)₁₋₁₀;
(ADP7; SEQ ID NO: 18)
(HPP(S/T)HTLQPHHH(L/I)PVVPAQ QPV(A/I)PQQPMMPVPG
(H/Q)HSMTP(T/I)QH)₁₋₁₀;
and
(ADP7; SEQ ID NO: 18)
12-42 contiguous amino acids of (HPP(S/T)HTLQPHHH
(L/I)PVVPAQQPV(A/I)PQQPMMPVPG(H/Q)HSMTP(T/I)
QH)₁₋₁₀;

or a functional equivalent thereof, or any combination thereof. In some embodiments, the biomineralizing polypeptide comprises the amino acid sequence (PGYINFSYENSHSQAINVDRTA)₁₋₁₀ (ADP5H; SEQ ID NO:15), or a functional equivalent thereof. In some embodiments, the biomineralizing polypeptide comprises the amino acid sequence (SYENSHSQAINVDRT)₁₋₁₀ (shADP5; SEQ ID NO:16), or a functional equivalent thereof. In some embodiments, the biomineralizing polypeptide comprises (HPP(S/T)HTLQPHHH(L/I)PVVPAQQPV(A/I)PQ QPMMPVPG(H/Q)HSMTP(T/I)QH)₁₋₁₀ (ADP7; SEQ ID NO:18), 12-42 contiguous amino acids of (HPP(S/T)HTLQPHHH(L/I) PVVPAQQPV(A/I)PQQPMMPVPG(H/Q)HSMTP (T/I)QH)₁₋₁₀ (ADP7; SEQ ID NO:18), or a functional equivalent thereof. In some embodiments, the biomineralizing polypeptide comprises an amino acid sequence selected from the group consisting of:

(ADP1)
(SEQ ID NO: 1)
(HTLQPHHH(L/I)PVV)₁₋₁₀;
(ADP2)
(SEQ ID NO: 4)
(VPG(H/Q)HSMTP(T/I)QH)₁₋₁₀
(ADP4)
(SEQ ID NO: 10)
(HPP(S/T)HTLQPHHH(L/I)PVV)₁₋₁₀;
(ADP8)
(SEQ ID NO: 21)
(PAQQPV(A/I)PQQPMMP)₁₋₁₀;
(ADP7)
(SEQ ID NO: 18)
(HPP(S/T)HTLQPHHH(L/I)PVVPAQQPV(A/I)PQQPMMPVP(H/Q)
HSMTP(T/I)QH)₁₋₁₀;
(ADP1M)
(SEQ ID NO: 2)
(HTLQPHHHLPVV)₁₋₁₀;
(ADP1H)
(SEQ ID NO: 3)
(HTLQPHHHIPVV)₁₋₁₀;
(ADP2M)
(SEQ ID NO: 5)
(VPGHHSMTPTQH)₁₋₁₀;
(ADP2H)
(SEQ ID NO: 6)
(VPGQHSMTPIQH)₁₋₁₀;
(ADP4M)
(SEQ ID NO: 11)
(HPPSHTLQPHHELPVV)₁₋₁₀;
(ADP4H)
(SEQ ID NO: 12)
(HPPTHTLQPHHHIPVV)₁₋₁₀;
(ADP7M)
(SEQ ID NO: 19)
(HPPSHTLQPHHELPVVPAQQPVAPQQPMMPVPGHHSMTPTQH)₁₋₁₀;
(ADP7H)
(SEQ ID NO: 20)
(HPPTHTLQPHHHIPVVPAQQPVIPQQPMMPVPGQHSMTPIQH)₁₋₁₀;
(ADP8M)
(SEQ ID NO: 22)
(PAQQPVAPQQPMMP)₁₋₁₀;
and
(ADP8H)
(SEQ ID NO: 23)
(PAQQPVIPQQPMMP)₁₋₁₀;

or a functional equivalent thereof, or any combination thereof. In some embodiments, the biomineralizing polypeptide comprises an amino acid sequence selected from the group consisting of:

(ADP3M)
(SEQ ID NO: 8)
(WPATDKTKREEVD)₁₋₁₀;
and
(ADP3H)
(SEQ ID NO: 9)
(WPSTDKTKREEVD)₁₋₁₀,

or a functional equivalent thereof, or a combination thereof. In some embodiments, the biomineralizing polypeptide comprises one or more fusion peptides, wherein each of the one or more fusion peptides independently comprises two or more amino acid sequences selected from the group consisting of:

(ADP1)
(SEQ ID NO: 1)
(HTLQPHHH(L/I)PVV)₁₋₁₀;
(ADP2)
(SEQ ID NO: 4)
(VPG(H/Q)HSMTP(T/I)QH)₁₋₁₀
(ADP3)
(SEQ ID NO: 7)
WP(A/S)TDKTKREEVD₁₋₁₀;
(ADP4)
(SEQ ID NO: 10)
(HPP(S/T)HTLQPHHH(L/I)PVV)₁₋₁₀;
(ADP5)
(SEQ ID NO: 13)
(PGYIN(L/F)SYE(K/N)SHSQAIN(T/V)DRTA)₁₋₁₀;
(ADP6)
(SEQ ID NO: 17)
(LPPLFSMPLSPILPELPLEAWPAT)₁₋₁₀;
(ADP8)
(SEQ ID NO: 21)
(PAQQPV(A/I)PQQPMMP)₁₋₁₀;
(ADP7)
(SEQ ID NO: 18)
(HPP(S/T)HTLQPHHH(L/I)PVVPAQQPV(A/I)PQQPMMPVP(H/Q)
HSMTP(T/I)QH)₁₋₁₀;
(ADP1M)
(SEQ ID NO: 2)
(HTLQPHHHLPVV)₁₋₁₀;
(ADP1H)
(SEQ ID NO: 3)
(HTLQPHHHIPVV)₁₋₁₀;
(ADP2M)
(SEQ ID NO: 5)
(VPGHHSMTPTQH)₁₋₁₀;
(ADP2H)
(SEQ ID NO: 6)
(VPGQHSMTPIQH)₁₋₁₀;
(ADP4M)
(SEQ ID NO: 11)
(HPPSHTLQPHHELPVV)₁₋₁₀;
(ADP4H)
(SEQ ID NO: 12)
(HPPTHTLQPHHHIPVV)₁₋₁₀;
(ADP7M)
(SEQ ID NO: 19)
(HPPSHTLQPHHELPVVPAQQPVAPQQPMMPVPGHHSMTPTQH)₁₋₁₀;
(ADP7H)
(SEQ ID NO: 20)
(HPPTHTLQPHHHIPVVPAQQPVIPQQPMMPVPGQHSMTPIQH)₁₋₁₀;
(ADP8M)
(SEQ ID NO: 22)
(PAQQPVAPQQPMMP)₁₋₁₀;
and
(ADP8H)
(SEQ ID NO: 23)
(PAQQPVIPQQPMMP)₁₋₁₀;

or a functional equivalent thereof. In some embodiments, the biomineralizing polypeptide further comprises a fluorescent agent.

### BRIEF DESCRIPTION OF THE DRAWINGS

The foregoing aspects and many of the attendant advantages of the described technology will become more readily appreciated as the same become better understood by reference to the following detailed description, when taken in conjunction with the accompanying drawings, wherein:

FIG. 1 depicts a tooth (a) before and (b) after sectioning vertically as described in Example 2.

FIGS. 2(a) to 2(g) depict various sections of a tooth after staining as described in Example 2. FIG. 2(a) shows one half of a sectioned tooth after the staining. FIGS. 2(b), (c) and (d) show different views of the left quarter of the tooth that was sectioned from the half tooth. FIGS. 2(e), (f) and (g) show different views of the right quarter of the tooth that was sectioned from the half tooth.

FIG. 3 shows the left quarter (control) and the right quarter of the tooth (test tooth) as described in Example 2.

FIG. 4 depicts a line profile analysis of a portion of the original tooth (before staining), a portion of the tooth after staining, and a portion of tooth after staining and remineralization, as described in Example 2.

FIG. 5 depicts a tooth before staining, a side-by-side comparison of a stained tooth portion with a 3-rounds remineralized tooth portion, a side-by-side comparison of a stained tooth portion with a 4-rounds remineralized tooth portion, a side-by-side comparison of a stained tooth portion with a 5-rounds remineralized tooth portion, and a side-by-side comparison of a stained tooth portion with a 7-rounds remineralized tooth portion, as described in Example 8.

FIG. 6 shows a line profile analysis of the tooth before staining (Bar A), the 3-rounds remineralized tooth (Bar B), the 4-rounds remineralized tooth (Bar C), the 5-rounds remineralized tooth (Bar D), and the 7-rounds remineralized tooth (Bar E), as described in Example 8.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the singular forms “a”, “an” and “the” include plural referents unless the context clearly dictates otherwise. “And” as used herein is interchangeably used with “or” unless expressly stated otherwise. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (for example, “such as”) provided herein, is intended merely to better illuminate the embodiments and does not pose a limitation on the scope of the claims unless otherwise stated. No language in the specification should be construed as indicating any non-claimed element as essential.

All embodiments of any aspect of the described technologies can be used in combination, unless the context clearly dictates otherwise.

In a first aspect, methods for whitening teeth are provided, comprising administering to a subject in need thereof an amount effective to whiten teeth of a biomineralizing polypeptide or pharmaceutical composition or oral care product of any embodiment or combination of embodiments of the technologies. The methods can further comprise administering at least one calcium ion source and at least one phosphate ion source to the subject. The calcium ion source and phosphate ion source can be co-administered or serially administered. The calcium ion source and phosphate ion source can be administered before the biomineralizing polypeptide, concurrently with the biomineralizing polypeptide, or after the biomineralizing polypeptide.

As shown herein, the inventors have discovered that biomineralizing polypeptides can be used to whiten teeth. While not being bound by a specific mechanism of action, the inventors believe that the polypeptides direct mineralization of dental lesions to form a “dento-mimetic” mineral layer, which also serves to reduce dental hypersensitivity and bacterial infiltration. It is further believed that some of the polypeptides exert their activity via binding to hydroxyapatite (HA) surfaces on the tooth to kinetically promote re-mineralization, occluding of dentin tubules to prevent/limit stimulants from reaching the tubules, and/or rebuilding lost mineral to create a physical barrier against bacteria. Thus, the described methods provide a great improvement over previously reported methods for treating dental disease.

The methods of the present application have a number of attendant advantages. The described methods clear stains present on tooth surfaces in addition to re-mineralizing tooth surfaces. Without clearing stains any re-mineralization would create incipient lesions on the tooth. The described methods lead to whitened and smooth tooth surfaces in contrast to conventional whiteners which may lead to uneven tooth surfaces, with “hills” and “valleys”.

As used herein, a “biomineralizing polypeptide” is any polypeptide capable of generating a hydroxyapatite layer on a tooth in the presence of calcium ions and phosphate ions. Several biomineralizing polypeptides are described in PCT application No. PCT/US2012/039650, which is incorporated herein by reference in its entirety. The calcium ions are present due to at least one calcium ion source. The calcium ion source can generally be any calcium salt. Illustrative calcium ion sources include, but are not limited to, calcium acetate, calcium carbonate, calcium citrate, calcium chloride, calcium gluconate, calcium glycerophosphate, calcium lactate, and calcium phosphate. In some cases, combinations of more than one calcium ion source may be used. In some embodiments, the calcium ion source is not calcium phosphate. The phosphate ions are present due to at least one phosphate ion source. The phosphate ion source can generally be any phosphate salt. Illustrative phosphate ion sources include, but are not limited to, aluminum phosphates, calcium phosphates, potassium phosphates, and sodium phosphates. Calcium phosphates include monocalcium phosphate, dicalcium phosphate, and tricalcium phosphate. Potassium phosphates include monopotassium phosphate, dipotassium phosphate, and tripotassium phosphate. Sodium phosphates include sodium dihydrogen phosphate, sodium hydrogen phosphate, and trisodium phosphate. In some cases, combinations of more than one phosphate ion source may be used. In some embodiments, the phosphate ion source is not calcium phosphate. The concentration of calcium ions can generally be any concentration, such as about 0.1 mM to about 100 mM. The concentration of phosphate ions can generally be any concentration, such as about 0.06 mM to about 60 mM. The ratio of calcium ions to phosphate ions can generally be any ratio, such as about 5:3.

In some embodiments, biomineralizing polypeptides comprise or consist of one or more amelogenin derived polypeptides (ADPs).

In one embodiment, biomineralizing polypeptides comprise or consist of

(ADP3; SEQ ID NO: 7)
(WP(A/S)TDKTKREEVD)₁₋₁₀;
(ADP5; SEQ ID NO: 13)
(PGYTN(L/F)SYE(K/N)SHSQAIN(T/V)DRTA)₁₋₁₀;
(ADP6; SEQ ID NO: 17)
(LPPLFSMPLSPILPELPLEAWPAT)₁₋₁₀;
(ADP7; SEQ ID NO: 18)
(HPP(S/T)HTLQPHHH(L/I)PVVPAQ QPV(A/I)PQQPMMPVPG
(H/Q)HSMTP(T/I)QH)₁₋₁₀;
and
(ADP7; SEQ ID NO: 18)
12-42 contiguous amino acids of (HPP(S/T)HTLQPHHH
(L/I)PVVPAQQPV(A/I)PQQPMMPVPG(H/Q)HSMTP(T/I)
QH)₁₋₁₀;

or functional equivalents thereof. In various embodiments, the recited polypeptides may be present in 1, 2, 3, 4, 5, 6, 7, 8, 9, or 10 copies, or ranges such as 2-10. For example, a tetrapeptide can be used that contains four consecutive copies of ADP6. In some embodiments, the biomineralizing polypeptides comprise or consist of two or more of the ADPs or functional equivalents thereof. In some embodiments, the biomineralizing polypeptides comprise one or more fusion peptides comprising or consisting of two or more of the ADPs or functional equivalents thereof.

As used herein, a “functional equivalent” of a polypeptide is one that retains the biological activity of the polypeptide in treating dental disease, and includes one or more amino acid substitutions, deletions, additions, or insertions. In various embodiments, the functional equivalent is at least 50%, 55%, 60%, 65%, 70%, 75%, 80%, 85%, 90%, 91%, 92%, 93%, 94%, 95%, or more identical to the recited polypeptide. In some embodiments, the functional equivalent is a shortened version of an ADP described herein.

As used throughout the present application, the term “polypeptide” is used in its broadest sense to refer to a sequence of subunit amino acids, whether naturally occurring or of synthetic origin. The polypeptides may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo, such as by PEGylation, HESylation, PASylation, or glycosylation. Such linkage can be covalent or non-covalent as is understood by those of skill in the art.

In one embodiment, the methods comprise administering a polypeptide comprising or consisting of the amino acid sequence (PGYIN(L/F)SYE(K/N)SHSQAIN(T/V)DRTA)₁₋₁₀ (ADP5; SEQ ID NO:13), or a functional equivalent thereof. In an alternative embodiment, ADP5 comprises or consists of (PGYINFSYENSHSQAINVDRTA)₁₋₁₀ (ADP5H; SEQ ID NO:15), or a functional equivalent thereof. In another alternative embodiment, ADP5 comprises or consists of (PGYINLSYEKSHSQAINTDRTA)₁₋₁₀ (ADP5M; SEQ ID NO:14), or a functional equivalent thereof. In yet another alternative embodiment, ADP5 comprises or consists of (SYENSHSQAINVDRT)₁₋₁₀ (shADP5; SEQ ID NO:16), or a functional equivalent thereof. In various embodiments, ADP5, ADP5M, shADP5 or ADP5H is present in 1, 2, 3, 4, 5, 6, 7, 8, 9, or 10 copies, or ranges such as 2-10. In an alternative embodiment, ADP5, ADP5M, shADP5 or ADP5H is present in 1 copy. In yet another alternative embodiment, the ADP5 comprises or consists of ADP5Hor shADP5, preferably in one copy. In some embodiments, the biomineralizing polypeptides comprise or consist of two or more of the ADPs or functional equivalents thereof. In some embodiments, the biomineralizing polypeptides comprise one or more fusion peptides comprising or consisting of two or more of the ADPs or functional equivalents thereof.

In another embodiment, the methods comprise administering a polypeptide comprising or consisting of the amino acid sequence:

(HPP(S/T)HTLQPHHH(L/I)PVVPAQQPV (A/I)PQQPMMPVPG(H/Q)HSMTP(T/I)QH)₁₋₁₀ (ADP7; SEQ ID NO:18), or a functional equivalent thereof. In an embodiment, the ADP7 comprises or consists of
(HPPSHTLQPHHHLPVVPAQQPVAPQQPMMPVPGHHSMTPTQH)₁₋₁₀ (ADP7M; SEQ ID NO:19); (HPPTHTLQPHHHIPVVPAQQPVIPQQPMMPVPGQHSMTPIQH)₁₋₁₀ (ADP7H; SEQ ID NO:20), or a functional equivalent thereof. In various embodiments, ADP7, ADP7M, or ADP7H is present in 1, 2, 3, 4, 5, 6, 7, 8, 9, or 10 copies, or ranges such as 2-10. In an embodiment, ADP7, ADP7M, or ADPH7 is present in 1 copy. In another embodiment, the ADP7 comprises or consists of ADP7H in one copy. In some embodiments, the biomineralizing polypeptides comprise or consist of two or more of the ADPs or functional equivalents thereof. In some embodiments, the biomineralizing polypeptides comprise one or more fusion peptides comprising or consisting of two or more of the ADPs or functional equivalents thereof.

In a further embodiment, the polypeptide for use in the methods comprises or consists of the amino acid sequence WP(A/S)TDKTKREEVD)₁₋₁₀ (ADP3; SEQ ID NO:7), or a functional equivalent thereof. In an embodiment, ADP3 comprises or consists of ((WPATDKTKREEVD)₁₋₁₀ (ADP3M; SEQ ID NO:8) or (WPSTDKTKREEVD)₁₋₁₀ (ADP3H; SEQ ID NO:9), or a functional equivalent thereof. In various embodiments, ADP3, ADP3M, or ADP3H is present in 1, 2, 3, 4, 5, 6, 7, 8, 9, or 10 copies, or ranges such as 2-10. In an embodiment, ADP3, ADP3M, or ADP3H is present in 1 copy. In another embodiment, the ADP3 comprises or consists of ADP3H in one copy. In some embodiments, the biomineralizing polypeptides comprise or consist of two or more of the ADPs or functional equivalents thereof. In some embodiments, the biomineralizing polypeptides comprise one or more fusion peptides comprising or consisting of two or more of the ADPs or functional equivalents thereof.

In various further embodiments, the polypeptide for use in the methods comprises or consists of a polypeptide selected from the group consisting of:

(ADP1)
(SEQ ID NO: 1)
(HTLQPHHH(L/I)PVV)₁₋₁₀;
(ADP2)
(SEQ ID NO: 4)
(VPG(H/Q)HSMTP(T/I)QH)₁₋₁₀
(ADP4)
(SEQ ID NO: 10)
(HPP(S/T)HTLQPHHH(L/I)PVV)₁₋₁₀;
(ADP8)
(SEQ ID NO: 21)
(PAQQPV(A/I)PQQPMMP)₁₋₁₀;
(ADP7)
(SEQ ID NO: 18)
(HPP(S/T)HTLQPHHH(L/I)PVVPAQQPV(A/I)PQQPMMPVP(H/Q)
HSMTP(T/I)QH)₁₋₁₀;
(ADP1M)
(SEQ ID NO: 2)
(HTLQPHHHLPVV)₁₋₁₀;
(ADP1H)
(SEQ ID NO: 3)
(HTLQPHHHIPVV)₁₋₁₀;
(ADP2M)
(SEQ ID NO: 5)
(VPGHHSMTPTQH)₁₋₁₀;
(ADP2H)
(SEQ ID NO: 6)
(VPGQHSMTPIQH)₁₋₁₀;
(ADP4M)
(SEQ ID NO: 11)
(HPPSHTLQPHHELPVV)₁₋₁₀;
(ADP4H)
(SEQ ID NO: 12)
(HPPTHTLQPHHHIPVV)₁₋₁₀;
(ADP7M)
(SEQ ID NO: 19)
(HPPSHTLQPHHELPVVPAQQPVAPQQPMMPVPGHHSMTPTQH)₁₋₁₀;
(ADP7H)
(SEQ ID NO: 20)
(HPPTHTLQPHHHIPVVPAQQPVIPQQPMMPVPGQHSMTPIQH)₁₋₁₀;
(ADP8M)
(SEQ ID NO: 22)
(PAQQPVAPQQPMMP)₁₋₁₀;
and
(ADP8H)
(SEQ ID NO: 23)
(PAQQPVIPQQPMMP)₁₋₁₀;

or functional equivalents thereof.

In various embodiments, the recited polypeptides may be present in 1, 2, 3, 4, 5, 6, 7, 8, 9, or 10 copies, or ranges such as 2-10. In some embodiments, when present in more than one copy, the copies are contiguous to each other. In some embodiments, the polypeptide for use in the methods comprises a fusion of two or more of the ADPs described herein or functional equivalents thereof.

Each ADP and functional equivalent thereof has its own distinct kinetic profile and may exhibit a fast or slow kinetic profile. For example, ADP5 has a fast kinetic profile whereas ADP7 has a slow kinetic profile. In some embodiments, the biomineralizing polypeptide comprises or consists of at least one ADP with a slow kinetic profile and at least one ADP with a fast kinetic profile. In some embodiments, the biomineralizing polypeptide only comprises or consists of one or more ADPs with fast kinetic profiles. In some embodiments, the biomineralizing polypeptide only comprises or consists of one or more ADPs with slow kinetic profiles. Fast kinetics refers to fast mineral formation, such as immediate mineral formation, while slow mineral formation refers to mineral formation over a period of time, such as about 5 minutes to about 1 hour. In some embodiments, a first biomineralizing peptide having faster kinetics can be used in combination with a second biomineralizing peptide having slower kinetics.

In some embodiments, the polypeptide for use in the methods further comprises at least one fluorescent agent. In some embodiments, the polypeptide for use in the methods comprises a polypeptide covalently linked to at least one fluorescent agent. Illustrative fluorescent agents include, but are not limited to, fluorescein, 6-FAM, rhodamine, Texas Red, California Red, iFluor594, tetramethylrhodamine, a carboxyrhodamine, carboxyrhodamine 6F, carboxyrhodol, carboxyrhodamine 110, Cascade Blue, Cascade Yellow, coumarin, Cy2®, Cy3®, Cy3.5®, Cy5®, Cy5.5®, Cy7®, Cy-Chrome, DyLight® 350, DyLight® 405, DyLight® 488, DyLight® 549, DyLight® 594, DyLight® 633, DyLight® 649, DyLight® 680, DyLight® 750, DyLight® 800, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and -6-)-carboxy-X-rhodamine), HEX, Lucifer Yellow, Marina Blue, Oregon Green 488, Oregon Green 500, Oregon Green 514, Alexa Fluor® 350, Alex Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br2, BODIPY® 530/550, BODIPY® 558/568, BODIPY® 630/650, BODIPY® 650/665, BODIPY® R6G, BODIPY® TMR, BODIPY® TR, and combinations thereof.

The subject may be any subject whose teeth could be whitened, including but not limited to mammals. In various embodiments, the mammal is a human, dog, cat, horse, cow, sheep, goat, pig, or other pet or food/dairy animal. In one embodiment, the subject is a human.

As used herein, “tooth whitening” and “dental bleaching” have identical meanings and can be used interchangeably. Tooth whitening and dental bleaching refer to decreasing the amount of chromagens or discolorants present in or on a tooth. In certain embodiments, tooth whitening refers to restoring a tooth to its original color. In certain other embodiments, dental bleaching refers to whitening a tooth beyond its natural, original color. Tooth whitening may be measured using methods known to those skilled in the art. In some embodiments, tooth whitening is measured using comparative shade guides. In some embodiments, tooth color and tooth whitening are measured using a colorimeter, such as a tristimulus colorimeter. Such instruments can be used to measure the color of the specimen (tooth) surface quantitatively. The CIE “L*a*b*” color system is commonly used in the commercial and scientific literature for quantitatively measuring teeth color (See Yiming, J. Esther Restor. Dent., 15: 533-541 (2003)). The system uses one luminance parameter and two color coordinates to specify a point on a chromaticity diagram. The “L” value corresponds to lightness, the “a” value corresponds to red-green, and the “b” value corresponds to yellow-blue. A change in the color of a treated tooth can be calculated as ΔELab=((ΔL*)²+(Δa*)²+(Δb*)²)^{1/2}. The value can generally be any value, and in some cases have a minimum value limited only by the detection limit of the instrument used to take the measurements.

As used herein, unless otherwise described, an “amount effective” or “effective amount” refers to an amount of the polypeptide that is effective for whitening teeth. In some embodiments, the “amount effective” or “effective amount” means a concentration of about 0.01 mM to about 1 mM of the polypeptide. This includes a concentration of about 0.05 mM to about 0.5 mM or about 0.1 mM to about 0.5 mM. In some embodiments, the concentration is about 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, or 1 mM, including increments therein, of polypeptide. In a clinical setting, higher concentrations of polypeptide (for example, about 0.6 mM to about 0.8 mM) may be used. In a home use setting, lower concentrations of polypeptide (for example, about 0.01 mM to about 0.1 mM) may be used. In some embodiments, the “amount effective” or “effective amount” refers to an amount of a pharmaceutical composition comprising the polypeptide that is effective for whitening teeth.

The polypeptides are typically formulated as a pharmaceutical composition, such as those disclosed herein, and can be administered via any suitable route, including orally, parentally, by inhalation spray, or topically in dosage unit formulations containing conventional pharmaceutically acceptable carriers, adjuvants, and vehicles. In an embodiment, the pharmaceutical compositions and formulations are topically administration, such as in the form of ointments, lotions, creams, pastes, gels, drops, sprays, liquids and powders. Conventional pharmaceutical carriers, aqueous, powder or oily bases, thickeners and the like may be included.

In certain preferred embodiments, the polypeptides are delivered by: placing the polypeptides in contact with a tooth surface and applying a light source to the tooth. Without being bound by theory, it is believed that a light source can increase the rate of tooth whitening. Such light sources can include diode lasers, PAC lights, and halogen lights.

In certain preferred embodiments, the polypeptides are delivered by placing the polypeptides in contact with a tooth surface also in the presence of at least one cleaning agent. Examples of cleaning agents include hydrogen peroxide, carbamide peroxide, titanium dioxide, nano-hydroxyapatite particles, zirconia powder, and combinations thereof.

In certain preferred embodiments, the biomineralizing polypeptides are present in a gel or other pharmaceutical composition, as described herein, placed in a mouth guard and applied to the teeth of a subject overnight. Such repeated overnight application of biomineralizing polypeptides can have especially beneficial whitening effects.

Similarly, the biomineralizing polypeptides described herein can be applied to the teeth of a subject as a mouthwash, toothpaste, or tooth varnish on a daily basis to similar effect.

In some embodiments, the pharmaceutical compositions used in the methods described herein comprise at least one biomineralizing polypeptide and a calcium ion source. In some embodiments, the pharmaceutical compositions used in the methods described herein comprise at least one biomineralizing polypeptide and a phosphate ion source. In some embodiments, the pharmaceutical compositions used in the methods described herein comprise at least one biomineralizing polypeptide, a calcium ion source, and a phosphate ion source.

Dosage regimens can be adjusted to provide the optimum desired response (for example, a therapeutic or prophylactic response). A suitable dosage range may, for instance, be about 0.1 μg/kg to about 100 mg/kg body weight; alternatively, it may be about 0.5 μg/kg to about 50 mg/kg; about 1 μg/kg to about 25 mg/kg, or about 5 μg/kg to about 10 mg/kg body weight. The polypeptides can be delivered in a single bolus, or may be administered more than once (for example, 2, 3, 4, 5, or more times) as determined by an attending physician.

In various other embodiments, the heterologous polypeptide provides added functionality, for example, when the fusion polypeptides are used to whiten teeth. Exemplary such heterologous polypeptides include, but are not limited to biomineralization-promoting polypeptides (that is, any polypeptides that are useful for controlling or promoting biomineralization. As will be understood by those of skill in the art, the recited heterologous polypeptide may comprise or consist of the full length protein, or functional polypeptides derived therefrom. Such heterologous polypeptides are known to those of skill in the art. A recombinant fusion protein can comprise the ADP polypeptide and at least one heterologous polypeptide.

In a further aspect, pharmaceutical compositions are provided, comprising one or more polypeptides, recombinant fusion proteins, or compositions with a pharmaceutically acceptable carrier. The pharmaceutical compositions can be used, for example, in the methods described herein. The pharmaceutical composition may comprise in addition to the polypeptide (a) at least one lyoprotectant; (b) at least one surfactant; (c) at least one bulking agent; (d) at least one tonicity adjusting agent; (e) at least one stabilizer; (f) at least one preservative; and/or (g) at least one buffer.

In some embodiments, the buffer in the pharmaceutical composition is a Tris buffer, a histidine buffer, a phosphate buffer, a citrate buffer, or an acetate buffer. The pharmaceutical composition may also include at least one lyoprotectant, for example, sucrose, sorbitol, or trehalose. In certain embodiments, the pharmaceutical composition includes at least one preservative, for example, benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, or various mixtures thereof. In other embodiments, the pharmaceutical composition includes at least one bulking agent, such as glycine. In yet other embodiments, the pharmaceutical composition includes at least one surfactant, for example, polysorbate-20, polysorbate-40, polysorbate-60, polysorbate-65, polysorbate-80, polysorbate-85, poloxamer-188, sorbitan monolaurate, sorbitan monopalmitate, sorbitan monostearate, sorbitan monooleate, sorbitan trilaurate, sorbitan tristearate, sorbitan trioleate, or any combination thereof. The pharmaceutical composition may also include at least one tonicity adjusting agent, for example, a compound that renders the formulation substantially isotonic or isoosmotic with human blood. Exemplary tonicity adjusting agents include sucrose, sorbitol, glycine, methionine, mannitol, dextrose, inositol, sodium chloride, arginine, and arginine hydrochloride. In other embodiments, the pharmaceutical composition additionally includes at least one stabilizer, for example, a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides may be the sole active agent in the pharmaceutical composition, or the composition may further comprise one or more other active agents suitable for an intended use, including but not limited to antimicrobial polypeptides (inhibiting bacterial infection), biomineralization-promoting polypeptides (that is, any polypeptides that are useful for controlling or promoting biomineralization), inorganic material-binding polypeptides, three-dimensional scaffold-forming polypeptides, collagen, chitosan, amphiphilic peptides, protein-binding polypeptides, enamelin-derived polypeptides, tuftelin-derived peptides, statherin-derived polypeptides, dentin-derived polypeptides, bone sialoprotein-derived polypeptides, osteocalcin-derived polypeptides, osteopontin-derived polypeptides, proteins with caries inhibitory activity, casein, and bone morphogenetic-derived polypeptides.

The pharmaceutical compositions described herein generally comprise a combination of a compound described herein and a pharmaceutically acceptable carrier, diluent, or excipient. Such compositions are substantially free of non-pharmaceutically acceptable components, that is, contain amounts of non-pharmaceutically acceptable components lower than permitted by regulatory requirements. In some embodiments of this aspect, if the compound is dissolved or suspended in water, the composition further optionally comprises an additional pharmaceutically acceptable carrier, diluent, or excipient. In other embodiments, the pharmaceutical compositions described herein are solid pharmaceutical compositions (for example, tablet, capsules, lozenges, and so on).

These compositions can be prepared in a manner well known in the pharmaceutical art, and can be administered by any suitable route. In an embodiment, the pharmaceutical compositions and formulations are designed for topical administration, and may include ointments, lotions, creams, pastes, gels, drops, sprays, liquids and powders. Conventional pharmaceutical carriers, aqueous, powder or oily bases, thickeners and the like may be necessary or desirable.

The pharmaceutical compositions can be in any suitable form, including but not limited to tablets, pills, powders, lozenges, sachets, cachets, elixirs, suspensions, emulsions, solutions, syrups, aerosols (as a solid or in a liquid medium), ointments containing, for example, up to about 10% by weight of the active compound, soft and hard gelatin capsules, sterile injectable solutions, and sterile packaged powders.

In certain embodiments, the pharmaceutical compositions comprise at least one cleaning agent in addition to the biomineralizing polypeptides. These cleaning agents can include hydrogen peroxide, carbamide peroxide, titanium dioxide, nano-hydroxyapatite particles, zirconia powder, or combinations thereof. The pharmaceutical compositions can also include abrasive agents, such as silica particles. In some embodiments, the pharmaceutical compositions further comprise a calcium ion source, a phosphate ion source, or both.

In one embodiment the pharmaceutical compositions are in the form of an oral care product, including but not limited to toothpaste, toothpowders, mouthwash, dental floss, liquid dentifrices, dental tablets, topical gels, troches, chewing gums, dental pastes, gingival massage creams, gargle tablets, lozenges, and food products. Thus, in another aspect, oral care products are provided, comprising any embodiment or combination of embodiments of the polypeptides, recombinant fusion proteins, and/or compositions. Such oral care products can be used, for example, in whitening teeth. In some embodiments, provided herein are oral care products comprising at least one biomineralizing polypeptide, at least one calcium ion source, at least one phosphate ion source, and at least one cleaning agent. In further embodiments, provided herein are oral care products comprising at least one biomineralizing polypeptide, calcium phosphate, and one or more of hydrogen peroxide, titanium dioxide, carbamide peroxide, nano-hydroxyapatite particles, and zirconia powder.

Several exemplary embodiments of the present application are summarized in the table below.

| Product Type | Formulation | Use area |
|---|---|---|
| Whitening gel with Fast | Gel | In-clinic |
| mineralizing Polypeptides | | Power Bleaching |
| Whitening gel with Fast | Gel | In-clinic |
| mineralizing Polypeptides | | Power Bleaching |
| & Laser light | | |
| Whitening gel with Fast | Gel | In-clinic |
| mineralizing Polypeptides | | Power Bleaching |
| & customized tray | | |
| Whitening gel with Regular | Gel | At-home |
| mineralizing Polypeptides | | fast Bleaching |
| & Laser light | | |
| Whitening gel with Regular | Gel | At-home |
| mineralizing Polypeptides | | fast Bleaching |
| & Laser light | | |
| Whitening gel with | Gel | At-home |
| Regular mineralizing | | fast Bleaching |
| Polypeptides & Tray | | |
| Whitening toothpaste with | Toothpaste | At-home |
| Regular mineralizing | | Daily Bleaching |
| Polypeptides | | |
| Whitening toothpaste with Mild | Toothpaste | At-home |
| mineralizing Polypeptides | | Daily Bleaching |
| Whitening toothpaste with Mild | Toothpaste | At-home |
| mineralizing Polypeptides & 2-3% | | Daily Bleaching |
| H₂O₂ | | |
| Whitening toothpaste with Mild | Toothpaste | At-home |
| mineralizing Polypeptides & 2-3% | | Daily Bleaching |
| carbamide peroxide | | |
| Whitening Toothpaste with Mild | Toothpaste | At-home |
| mineralizing Polypeptides | | Daily Bleaching |
| Whitening solution with Mild | Mouthwash | At-home |
| mineralizing Polypeptides | | Daily Bleaching |
| Whitening solution with Mild | Mouthwash | At-home |
| mineralizing Polypeptides | | Daily Bleaching |
| & chimeric AMPs (separate) | | |

As used in this table, “fast” refers to a rapid single shot whitening treatment, “regular” refers to a clinically viable chairside treatment time period, and “mild” refers to over the counter consumer products.

All of these aspects/embodiments disclosed herein can be combined with any other aspect/embodiment, unless the context clearly dictates otherwise.

### EXAMPLES

### Example 1. Polypeptide Synthesis

rM180 amelogenin was created as described previously by Moradian-Oldak et al. (J. Struct. Biol., 2000, 131(1):27-37). The ADPs were synthesized by standard solid phase peptide synthesis technique on Wang resin using Fmoc chemistry and HBTU activation. CSBio 336s (CSBio, Menlo Park, Calif., USA) automated peptide synthesizer was used for the synthesis. The resulting resin-bound polypeptides were cleaved and side-chain-deprotected using Reagent K (trifluoroacetic acid/thioanisole/H₂O/phenol/ethanedithiol (87.5:5:5:2.5)) and, precipitated by cold ether. The crude polypeptides obtained were purified by reverse phase high performance liquid chromatography up to a >98% purity (Gemini 10μ C18 110A column). The masses of the purified polypeptides were checked by mass spectroscopy using a MALDI-TOF mass spectrometer (Bruker Daltonics, Billerica, Mass., USA).

### Example 2. Experimental In Vitro Procedures of Re-Mineralization Using the Peptide Solution on Rat Tooth

Rat teeth (molars) were obtained from sacrificed rats (for example, Sprague Dawley rats, age 3 weeks or older) from animal facility, collected, and stored in 100% alcohol. Prior to the re-mineralization experiments the teeth were stored in 1% bleach for 24 hours. The teeth did not have soft tissues or plaque/tartar. The teeth were then dried in an incubator in air at 37° C. for 3-6 hours to produce dry surface. The root site and section the crown were removed vertically using a diamond blade. Pictures were taken with camera attached to optical microscope. Exposed dentine sites in the one half of the sectioned tooth were etched with 35% phosphoric acid (Ultraetch™) for 60 seconds (to facilitate the stain intake). The etching gel was removed by rinsing the sample with distilled water for 3 minutes. The staining solution was prepared by boiling 2 g of tea in 100 ml of deionized water for 5 minutes, and then cooled down to room temperature and filtered. The tooth sample was soaked in the staining solution and incubated at 37° C. at 150 rpm for 24 hours. The tooth was removed from the staining solution and sonicated for 30 seconds in deionized water (sonic bath) to remove the excess amount of tea particles attached to tooth extrinsically. The one half of the sectioned tooth was cut vertically into half again to form two quarter sections of the tooth. A second set of pictures were taken with camera attached to optical microscope. One of the quarter pieces was stored in 24 mM Tris Buffer (pH: 7.4) for control and the other one was used for in vitro re-mineralization. The portion used for in vitro re-mineralization was submerged into peptide solution (600 μl) containing a mineralization polypeptide in a 48 well plate and incubated for 10 minutes at 37° C. After incubation, the mineralized tooth was placed in a new well. 400 μl of 9.6 mM CaCl₂ and 400 μl of 5.6 mM KH₂PO₄ were added to the well and quickly mixed with a pipetting the solution up and down. The tooth was incubated in the mineralization solution for 16 hours at 37° C. The mineralization polypeptide was SYENSHSQAINVDRT (shADP5; SEQ ID NO:16). The quarter portion of the tooth which had been exposed to the polypeptide solution, calcium ion source and phosphate ion source was observed to be lighter in color than the control.

In certain experiments, mineralization was repeated with a fresh polypeptide and mineralization solution for the second round of re-mineralization.

### Example 3. Experimental In Vitro Procedures of Re-Mineralization Using the Peptide Solution on Human Tooth

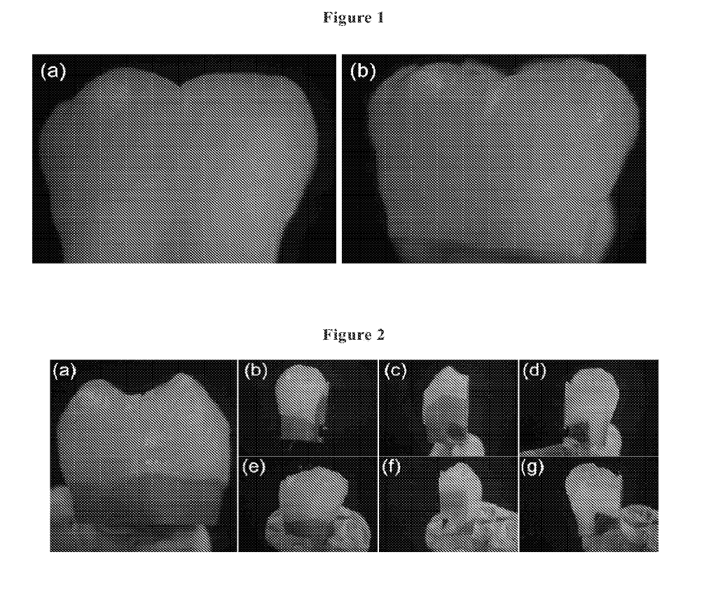

A caries-free human tooth was sterilized in 1% bleach solution for 24 hours then removed and vigorously rinsed with deionized water. The root site was removed and the crown sectioned vertically using a diamond blade. Pictures were taken with a camera attached to an optical microscope. FIG. 1(a) shows the original tooth before sectioning vertically. FIG. 1(b) shows one half of the original tooth that was sectioned.

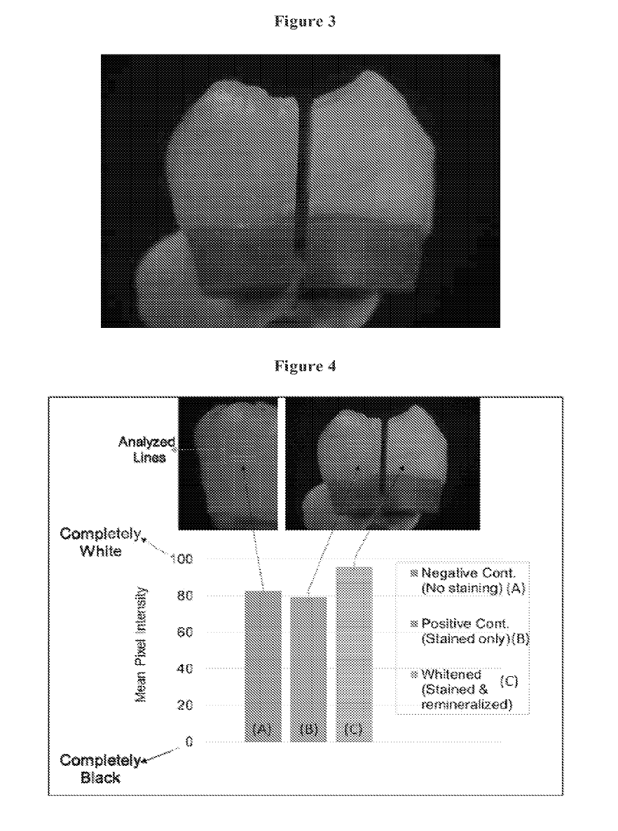

The exposed dentine sites were etched with 35% phosphoric acid (Ultraetch™) for 60 seconds (to facilitate the stain intake). The etching gel was removed by rinsing the sample with distilled water for 3 minutes. The staining solution was prepared by boiling 2 g of tea in 100 ml of deionized water for 5 minutes, which was then cooled down to room temperature and filtered. The tooth was soaked in the staining solution and incubated at 37° C. at 150 rpm for 24 hours. The tooth was then removed from the staining solution and sonicated for 30 seconds in deionized water (sonic bath) to remove the excess amount of tea particles attached to tooth extrinsically. The sectioned tooth was cut vertically in half again to form two quarter sections of the tooth. FIGS. 2(a) to 2(g) show various sections of the tooth after the staining. FIG. 2(a) shows the one half of the sectioned tooth after the staining. Figures (b), (c) and (d) show different views of the left quarter of the tooth that was sectioned from the tooth. Figures (e), (f) and (g) show different views of the right quarter of the tooth that was sectioned from the tooth. As can be observed from FIGS. 2(a) to 2(g), brown stains from the staining solution were formed on the surface and below the surface of the tooth. The left quarter of the tooth was stored in 24 mM Tris Buffer (pH: 7.4) for control and the right quarter of the tooth (test tooth) was used for in vitro re-mineralization. The test tooth was submerged into peptide solution (600 μl) containing a mineralization polypeptide in a 48 well plate and incubated for 10 minutes at 37° C. After incubation, the tooth was removed and placed into to a new well. A mineralization solution of 400 μl of 9.6 mM CaCl₂ and 400 μl of 5.6 mM KH₂PO₄ was added and quickly mixed by pipetting the solution up and down. The test tooth was incubated in the mineralization solution for 16 hours at 37° C. The mineralization polypeptide used was SYENSHSQAINVDRT (shADP5; SEQ ID NO:16). FIG. 3 shows the left quarter (control) and the right quarter of the tooth (test tooth). As can be seen in FIG. 3, the right quarter which had been exposed to the polypeptide solution, calcium ion source and phosphate ion source appeared lighter in color than the left quarter (control). In certain experiments, mineralization was repeated with a fresh polypeptide and mineralization solution for the second round of re-mineralization.

FIG. 4 shows a line profile analysis of a portion of the original tooth (before staining), a portion of the tooth after staining, and a portion of tooth after staining and remineralization. The bar chart in FIG. 4 shows the Mean Pixel Intensity (MPI) analyzed using ImageJ™ software on images of the tooth at before staining, after staining, and after staining and remineralization. A zero MPI indicates completely black while one hundred MPI indicates completely white. As can be observed from FIG. 4, the portion of the tooth that was stained and had subsequently undergone the remineralization treatment showed a higher MPI than the original tooth and the tooth after staining. Therefore, the remineralization treatment with the polypeptide solution and ion sources resulted in improved whiteness over the original tooth and the stained tooth.

| Assay | Mean Pixel Intensity | Bar in FIG. 4 |
|---|---|---|
| Negative control (no | 82.92 ± 1.37 | A |
| staining) | | |
| Positive control (stained | 79.24 ± 2.45 | B |
| only) | | |
| Whitened (stained and | 96.21 ± 2.39 | C |
| remineralized) | | |

### Example 4. Mineralization Polypeptide Solution Formulation

An exemplary solution formulation of a mineralization polypeptide described herein consists of Tris buffer pH 7.4, CaCl₂ as the calcium ion source, KH₂PO₄ as the phosphate ion source, and the mineralization polypeptide.

### Example 5. Mineralization Polypeptide Gel Formulation

An exemplary gel formulation of a mineralization polypeptide described herein is as follows:

| | |
|---|---|
| Ingredient | Weight % |

Solvent:

| | |
|---|---|
| Aqueous 1.6 mM shADP5 polypeptide | 50 |
| Aqueous 960 mM CaCl₂ | |
| Aqueous 560 mM K₂HPO₄ | |

Preservative:

| | |
|---|---|
| Potassium sorbate | 0.5 |

Thickener/humectant:

| | |
|---|---|
| Propylene glycol (PPG) | 3 |
| Glycerol (glycerin) | 15 |
| Cellulose Gum | 1 |

Sweetener/flavor:

| | |
|---|---|
| sorbitol (60% aqueous solution) | 30 |

Surfactant:

| | |
|---|---|
| simethicone (antifoam) | 0.5 |

### Example 6. Mineralization Polypeptide Tablet Formulation

An exemplary tablet formulation of a mineralization polypeptide described herein is as follows:

| | |
|---|---|
| Component | Weight (mg) |

Active Ingredient:

| | |
|---|---|
| mineralization polypeptide, calcium ions, phosphate ions | 300 |

Diluent:

| | |
|---|---|
| spray-dried lactose (SuperTab ® 11SD) | 88 |

Lubricant:

| | |
|---|---|
| magnesium stearate | 6 |

Glidant:

| | |
|---|---|
| talc | 6 |

### Example 7. Solution Mineralization Approach

The root site of an extracted human tooth was removed and the crown portion of the tooth was sectioned vertically into half. A thin layer of enamel was removed from one half of the sectioned tooth such that the dentin layer is exposed without completely removing the enamel on the tooth. This allowed imaging of the enamel surfaces after the mineralization experiment without the problem of surface reflection, and provides a more parallel surface from which to compare the whiteness results. The sectioned tooth was fixed onto a glass slide with epoxy. Initial color measurements of each of the four sides of the sectioned tooth was obtained using a Minolta Chroma Meter CR-200 (Konica Minolta, Tokyo, Japan), according to manufacturer instructions, using a white background and a black background. The sectioned tooth was incubated in a mineralization polypeptide solution for 10 minutes at 37° C. and then in a solution with calcium ions and phosphate ions for 2 hours at 37° C. Color measurements of each of the four sides of the sample were taken as described initially. The sectioned was rinsed with deionized water and the remineralization process (incubation in polypeptide solution, incubation in solution of ion sources, and rinsing with deionized water) was repeated for a total of 25 rounds. The mineralization polypeptide was SYENSHSQAINVDRT (shADP5; SEQ ID NO:16). Results of the experiment showed that whiteness of the tooth enamel improved with each round of remineralization.

### Example 8. Solution Mineralization Approach

Human teeth, donated by the University of Washington School of Dentistry (Seattle, Wash., USA), were selected for their clean, smooth surfaces. The teeth were sterilized in bleach, cut on a diamond blade to remove the root, and halved. A thin layer of the enamel surface was then cut on one of the halves parallel to the previous cut, such that the dentin layer could be seen without completely removing the enamel on the tooth. This allowed imaging of the enamel surfaces after the mineralization experiment without the problem of surface reflection encountered in previous experiments, in addition to a more parallel surface from which to compare the brightness or whiteness results. The cut enamel side of the tooth sample was then sonicated in DI water and polished on diamond slurry smooth out the surface for imaging. The sample was then cut into quarters, with the lower half selected for a tetracycline experiment because of the even distribution of dentin coloring in the lower quarters of the tooth sample. The samples were then stained in 10 mg/mL tetracycline solution for five days.

During mineralization, using a 96-well plate, one of the stained halves was stored in 1200 μL Tris (24 mM) as a negative control, and the experimental sample was incubated in 150 μL shADP5 (0.8 mM) at 37° C. for 10 minutes. The experimental sample was then rinsed of excess peptide with deionized water and then stored in 600 μL CaCl₂ (9.6 mM), to which 600 μL monobasic KH₂PO₄ (5.76 mM) was added. The well plate containing both the control and experimental samples was then stored at 37° C. for about 24 hours for mineralization to occur on the treated stained tooth sample.

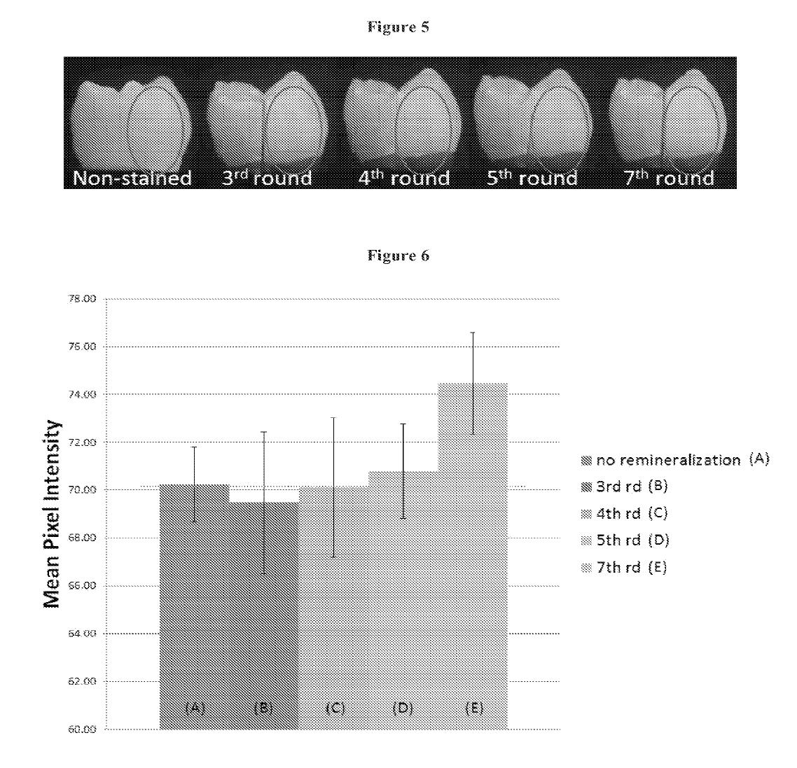

The remineralization process (incubation in polypeptide solution, incubation in solution of ion sources, and rinsing with deionized water) was repeated on the experimental sample for a total of 27 rounds. After 27 rounds of mineralization on the experimental sample, the tooth was inspected for changes in brightness qualitatively. The stained negative control was polished to expose the original enamel stain in order to compare the whitening effect of the peptide mineralization. FIG. 5 shows pictures of the tooth before staining, a side-by-side comparison of the stained negative control sample with the experimental sample having three rounds of remineralization, a side-by-side comparison of the stained negative control sample with the experimental sample having four rounds of remineralization, a side-by-side comparison of the stained negative control sample with the experimental sample having five rounds of remineralization, and a side-by-side comparison of the stained negative control sample with the experimental sample having seven rounds of remineralization. FIG. 6 shows a line profile analysis of the tooth before staining (Bar A), the 3-rounds remineralized tooth (Bar B), the 4-rounds remineralized tooth (Bar C), the 5-rounds remineralized tooth (Bar D), and the 7-rounds remineralized tooth (Bar E). The bar chart in FIG. 6 shows the Mean Pixel Intensity (MPI) of the tooth at the various stages.

The results were as follows, showing effective whitening from use of the biomineralization peptide:

| Assay | Mean Pixel Intensity | Bar in FIG. 6 |
|---|---|---|
| No staining or remineralization | 70.22 ± 1.57 | A |
| After 3 rounds | 69.47 ± 2.96 | B |
| After 4 rounds | 70.11 ± 2.92 | C |
| After 5 rounds | 70.79 ± 1.96 | D |
| After 7 rounds | 74.47 ± 2.13 | E |

As can be observed from bars (B) to (E) in FIG. 6 and the mean pixel intensity values in the table above, whiteness of the stained tooth improves with repeated rounds of remineralization.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

Unless otherwise indicated, to the extent that definitions of terms used herein differ between this application and references that have been incorporated herein in their entirety, the definitions of terms used herein presented in this application take precedence over the definitions presented in the references incorporated herein.

Other embodiments are set forth in the following claims.

1. A method for whitening teeth, comprising administering to a subject in need thereof an effective amount of a pharmaceutical composition comprising a biomineralizing polypeptide.

2. The method of claim 1, wherein the biomineralizing polypeptide comprises an amino acid sequence selected from the group consisting of:
(ADP3; SEQ ID NO: 7)
(WP(A/S)TDKTKREEVD)₁₋₁₀;
(ADP5; SEQ ID NO: 13)
(PGYTN(L/F)SYE(K/N)SHSQAIN(T/V)DRTA)₁₋₁₀;
(ADP6; SEQ ID NO: 17)
(LPPLFSMPLSPILPELPLEAWPAT);
(ADP7; SEQ ID NO: 18)
(HPP(S/T)HTLQPHHH(L/I)PVVPAQ QPV(A/I)PQQPMMPVPG
(H/Q)HSMTP(T/I)QH)₁₋₁₀;
and
(ADP7; SEQ ID NO: 18)
12-42 contiguous amino acids of (HPP(S/T)HTLQPHHH
(L/I)PVVPAQQPV(A/I)PQQPMMPVPG(H/Q)HSMTP(T/I)
QH)₁₋₁₀;
or a functional equivalent thereof, or any combination thereof.

3. The method of claim 2, wherein the biomineralizing polypeptide comprises the amino acid sequence (PGYIN(L/F)SYE(K/N)SHSQAIN(T/V)DRTA)₁₋₁₀ (ADP5; SEQ ID NO:13), or a functional equivalent thereof.

4. The method of claim 2, wherein the biomineralizing polypeptide comprises the amino acid sequence (PGYINFSYENSHSQAINVDRTA)₁₋₁₀ (ADP5H; SEQ ID NO:15), or a functional equivalent thereof.

5. The method of claim 2, wherein the biomineralizing polypeptide comprises amino acid sequence (SYENSHSQAINVDRT)₁₋₁₀ (shADP5; SEQ ID NO:16), or a functional equivalent thereof.

6. The method of claim 2, wherein the biomineralizing polypeptide comprises
(HPP(S/T)HTLQPHHH(L/I)PVVPAQQPV(A/I)PQQPMMPVPG(H/Q)HSMTP (T/I)QH)₁₋₁₀ (ADP7; SEQ ID NO:18),
12-42 contiguous amino acids of (HPP(S/T)HTLQPHHH(L/I)PVVPAQQPV(A/I) PQQPMMPVPG(H/Q)HSMTP(T/I)QH)₁₋₁₀ (ADP7; SEQ ID NO:18),
or a functional equivalent thereof.

7. The method of claim 6, wherein the biomineralizing polypeptide comprises an amino acid sequence selected from the group consisting of:
(ADP1)
(SEQ ID NO: 1)
(HTLQPHHH(L/I)PVV)₁₋₁₀;
(ADP2)
(SEQ ID NO: 4)
(VPG(H/Q)HSMTP(T/I)QH)₁₋₁₀
(ADP4)
(SEQ ID NO: 10)
(HPP(S/T)HTLQPHHH(L/I)PVV)₁₋₁₀;
(ADP8)
(SEQ ID NO: 21)
(PAQQPV(A/I)PQQPMMP)₁₋₁₀;
(ADP7)
(SEQ ID NO: 18)
(HPP(S/T)HTLQPHHH(L/I)PVVPAQQPV(A/I)PQQPMMPVP(H/Q)
HSMTP(T/I)QH)₁₋₁₀;
(ADP1M)
(SEQ ID NO: 2)
(HTLQPHHHLPVV)₁₋₁₀;
(ADP1H)
(SEQ ID NO: 3)
(HTLQPHHHIPVV)₁₋₁₀;
(ADP2M)
(SEQ ID NO: 5)
(VPGHHSMTPTQH)₁₋₁₀;
(ADP2H)
(SEQ ID NO: 6)
(VPGQHSMTPIQH)₁₋₁₀;
(ADP4M)
(SEQ ID NO: 11)
(HPPSHTLQPHHELPVV)₁₋₁₀;
(ADP4H)
(SEQ ID NO: 12)
(HPPTHTLQPHHHIPVV)₁₋₁₀;
(ADP7M)
(SEQ ID NO: 19)
(HPPSHTLQPHHELPVVPAQQPVAPQQPMMPVPGHHSMTPTQH)₁₋₁₀;
(ADP7H)
(SEQ ID NO: 20)
(HPPTHTLQPHHHIPVVPAQQPVIPQQPMMPVPGQHSMTPIQH)₁₋₁₀;
(ADP8M)
(SEQ ID NO: 22)
(PAQQPVAPQQPMMP)₁₋₁₀;
and
(ADP8H)
(SEQ ID NO: 23)
(PAQQPVIPQQPMMP)₁₋₁₀;
or a functional equivalent thereof, or a combination thereof.

8. The method of claim 2, wherein the biomineralizing polypeptide comprises an amino acid sequence selected from the group consisting of:
(WPATDKTKREEVD)₁₋₁₀ (ADP3M) (SEQ ID NO:8); and
(WPSTDKTKREEVD)₁₋₁₀ (ADP3H) (SEQ ID NO:9),
or a functional equivalent thereof, or a combination thereof.

9. The method of claim 1, wherein the biomineralizing polypeptide comprises one or more fusion peptides, wherein each of the one or more fusion peptides independently comprises two or more amino acid sequences selected from the group consisting of:
(ADP1)
(SEQ ID NO: 1)
(HTLQPHHH(L/I)PVV)₁₋₁₀;
(ADP2)
(SEQ ID NO: 4)
(VPG(H/Q)HSMTP(T/I)QH)₁₋₁₀
(ADP3)
(SEQ ID NO: 7)
WP(A/S)TDKTKREEVD₁₋₁₀;
(ADP4)
(SEQ ID NO: 10)
(HPP(S/T)HTLQPHHH(L/I)PVV)₁₋₁₀;
(ADP5)
(SEQ ID NO: 13)
(PGYIN(L/F)SYE(K/N)SHSQAIN(T/V)DRTA)₁₋₁₀;
(ADP6)
(SEQ ID NO: 17)
(LPPLFSMPLSPILPELPLEAWPAT)₁₋₁₀;
(ADP8)
(SEQ ID NO: 21)
(PAQQPV(A/I)PQQPMMP)₁₋₁₀;
(ADP7)
(SEQ ID NO: 18)
(HPP(S/T)HTLQPHHH(L/I)PVVPAQQPV(A/I)PQQPMMPVP(H/Q)
HSMTP(T/I)QH)₁₋₁₀;
(ADP1M)
(SEQ ID NO: 2)
(HTLQPHHHLPVV)₁₋₁₀;
(ADP1H)
(SEQ ID NO: 3)
(HTLQPHHHIPVV)₁₋₁₀;
(ADP2M)
(SEQ ID NO: 5)
(VPGHHSMTPTQH)₁₋₁₀;
(ADP2H)
(SEQ ID NO: 6)
(VPGQHSMTPIQH)₁₋₁₀;
(ADP4M)
(SEQ ID NO: 11)
(HPPSHTLQPHHELPVV)₁₋₁₀;
(ADP4H)
(SEQ ID NO: 12)
(HPPTHTLQPHHHIPVV)₁₋₁₀;
(ADP7M)
(SEQ ID NO: 19)
(HPPSHTLQPHHELPVVPAQQPVAPQQPMMPVPGHHSMTPTQH)₁₋₁₀;
(ADP7H)
(SEQ ID NO: 20)
(HPPTHTLQPHHHIPVVPAQQPVIPQQPMMPVPGQHSMTPIQH)₁₋₁₀;
(ADP8M)
(SEQ ID NO: 22)
(PAQQPVAPQQPMMP)₁₋₁₀;
and
(ADP8H)
(SEQ ID NO: 23)
(PAQQPVIPQQPMMP)₁₋₁₀;
or a functional equivalent thereof.

10. The method of claim 1, wherein the biomineralizing polypeptide further comprises a fluorescent agent.

11. The method of claim 1, further comprising applying light to the teeth of the subject, thereby further whitening the teeth, wherein the light is selected from one of a diode laser; a PAC light; and a halogen light.

12. The method of claim 1, further comprising administering at least one cleaning agent to the teeth of the subject, wherein the at least one cleaning agent is selected from the group consisting of: hydrogen peroxide, carbamide peroxide, titanium dioxide, nano-hydroxyapatite particles, zirconia powder, and any combination thereof.

13. (canceled)

14. The method of claim 1, wherein the biomineralizing polypeptide is present in the pharmaceutical composition at a concentration of about 0.01 mM to about 1 mM.

15. The method of claim 1, comprising administering to the subject the pharmaceutical composition further comprising at least one calcium ion source and at least one phosphate ion source.

16. (canceled)

17. An oral care product, comprising a biomineralizing polypeptide, at least one calcium ion source, and at least one phosphate ion source.

18. The oral care product of claim 17, further comprising at least one cleaning agent, wherein the at least one cleaning agent is selected from the group consisting of hydrogen peroxide, titanium dioxide, carbamide peroxide, nano-hydroxyapatite particles, zirconia powder, and any combination thereof.

19. (canceled)

20. The oral care product of claim 17, wherein the calcium ion source is selected from the group consisting of calcium acetate, calcium carbonate, calcium citrate, calcium chloride, calcium gluconate, calcium glycerophosphate, calcium lactate, and calcium phosphate.

21. The oral care product of claim 17, wherein the phosphate ion source is selected from the group consisting of aluminum phosphates, calcium phosphates, potassium phosphates, and sodium phosphates.

22. The oral care product of claim 17, wherein the oral care product is selected from the group consisting of toothpaste, toothpowders, mouthwash, gel, dental floss, liquid dentifrices, dental tablets, topical gels, troches, chewing gums, dental pastes, gingival massage creams, gargle tablets, lozenges, tooth trays, tooth varnishes, and food products.

23. The oral care product of claim 17, wherein the biomineralizing polypeptide comprises an amino acid sequence selected from the group consisting of:
(ADP3; SEQ ID NO: 7)
(WP(A/S)TDKTKREEVD)₁₋₁₀;
(ADP5; SEQ ID NO: 13)
(PGYTN(L/F)SYE(K/N)SHSQAIN(T/V)DRTA)₁₋₁₀;
(ADP6; SEQ ID NO: 17)
(LPPLFSMPLSPILPELPLEAWPAT) (Original);
(ADP7; SEQ ID NO: 18)
(HPP(S/T)HTLQPHHH(L/I)PVVPAQ QPV(A/I)PQQPMMPVPG
(H/Q)HSMTP(T/I)QH)₁₋₁₀;
and
(ADP7; SEQ ID NO: 18)
12-42 contiguous amino acids of (HPP(S/T)HTLQPHHH
(L/I)PVVPAQQPV(A/I)PQQPMMPVPG(H/Q)HSMTP(T/I)
QH)₁₋₁₀;
or a functional equivalent thereof, or any combination thereof.

24. The oral care product of claim 23, wherein the biomineralizing polypeptide comprises the amino acid sequence (PGYIN(L/F)SYE(K/N)SHSQAIN(T/V)DRTA)₁₋₁₀ (ADP5) (SEQ ID NO:13), or a functional equivalent thereof.

25. The oral care product of claim 23, wherein the biomineralizing polypeptide comprises the amino acid sequence (PGYINFSYENSHSQAINVDRTA)₁₋₁₀ (ADP5H; SEQ ID NO:15), or a functional equivalent thereof.

26. The oral care product of claim 23, wherein the biomineralizing polypeptide comprises the amino acid sequence (SYENSHSQAINVDRT)₁₋₁₀ (shADP5; SEQ ID NO:16), or a functional equivalent thereof.

27. The oral care product of claim 23, wherein the biomineralizing polypeptide comprises:
(ADP7; SEQ ID NO: 18)
(HPP(S/T)HTLQPHHH(L/I)PVVPAQQPV(A/I)PQQPMMPVPG
(H/Q)HSMTP(T/I)QH)₁₋₁₀,
(ADP7; SEQ ID NO: 18)
12-42 contiguous amino acids of (HPP(S/T)
HTLQPHHH(L/I)PVVPAQQPV(A/I)PQQPMMPVPG(H/Q)HSMTP
(T/I)QH)₁₋₁₀,
or a functional equivalent thereof.

28. The oral care product of claim 27, wherein the biomineralizing polypeptide comprises an amino acid sequence selected from the group consisting of:
(ADP1)
(SEQ ID NO: 1)
(HTLQPHHH(L/I)PVV)₁₋₁₀;
(ADP2)
(SEQ ID NO: 4)
(VPG(H/Q)HSMTP(T/I)QH)₁₋₁₀
(ADP4)
(SEQ ID NO: 10)
(HPP(S/T)HTLQPHHH(L/I)PVV)₁₋₁₀;
(ADP8)
(SEQ ID NO: 21)
(PAQQPV(A/I)PQQPMMP)₁₋₁₀;
(ADP7)
(SEQ ID NO: 18)
(HPP(S/T)HTLQPHHH(L/I)PVVPAQQPV(A/I)PQQPMMPVP(H/Q)
HSMTP(T/I)QH)₁₋₁₀;
(ADP1M)
(SEQ ID NO: 2)
(HTLQPHHHLPVV)₁₋₁₀;
(ADP1H)
(SEQ ID NO: 3)
(HTLQPHHHIPVV)₁₋₁₀;
(ADP2M)
(SEQ ID NO: 5)
(VPGHHSMTPTQH)₁₋₁₀;
(ADP2H)
(SEQ ID NO: 6)
(VPGQHSMTPIQH)₁₋₁₀;
(ADP4M)
(SEQ ID NO: 11)
(HPPSHTLQPHHELPVV)₁₋₁₀;
(ADP4H)
(SEQ ID NO: 12)
(HPPTHTLQPHHHIPVV)₁₋₁₀;
(ADP7M)
(SEQ ID NO: 19)
(HPPSHTLQPHHELPVVPAQQPVAPQQPMMPVPGHHSMTPTQH)₁₋₁₀;
(ADP7H)
(SEQ ID NO: 20)
(HPPTHTLQPHHHIPVVPAQQPVIPQQPMMPVPGQHSMTPIQH)₁₋₁₀;
(ADP8M)
(SEQ ID NO: 22)
(PAQQPVAPQQPMMP)₁₋₁₀;
and
(ADP8H)
(SEQ ID NO: 23)
(PAQQPVIPQQPMMP)₁₋₁₀;
or a functional equivalent thereof, or a combination thereof.

29. The oral care product of claim 23, wherein the biomineralizing polypeptide comprises an amino acid sequence selected from the group consisting of:
(ADP3M)
(SEQ ID NO: 8)
(WPATDKTKREEVD)₁₋₁₀;
and
(ADP3H)
(SEQ ID NO: 9)
(WPSTDKTKREEVD)₁₋₁₀,
or a functional equivalent thereof, or a combination thereof.

30. The oral care product of claim 17, wherein the biomineralizing polypeptide comprises one or more fusion peptides, wherein each of the one or more fusion peptides independently comprises two or more amino acid sequences selected from the group consisting of:
(ADP1)
(SEQ ID NO: 1)
(HTLQPHHH(L/I)PVV)₁₋₁₀;
(ADP2)
(SEQ ID NO: 4)
(VPG(H/Q)HSMTP(T/I)QH)₁₋₁₀
(ADP3)
(SEQ ID NO: 7)
WP(A/S)TDKTKREEVD₁₋₁₀;
(ADP4)
(SEQ ID NO: 10)
(HPP(S/T)HTLQPHHH(L/I)PVV)₁₋₁₀;
(ADP5)
(SEQ ID NO: 13)
(PGYIN(L/F)SYE(K/N)SHSQAIN(T/V)DRTA)₁₋₁₀;
(ADP6)
(SEQ ID NO: 17)
(LPPLFSMPLSPILPELPLEAWPAT)₁₋₁₀;
(ADP8)
(SEQ ID NO: 21)
(PAQQPV(A/I)PQQPMMP)₁₋₁₀;
(ADP7)
(SEQ ID NO: 18)
(HPP(S/T)HTLQPHHH(L/I)PVVPAQQPV(A/I)PQQPMMPVP(H/Q)
HSMTP(T/I)QH)₁₋₁₀;
(ADP1M)
(SEQ ID NO: 2)
(HTLQPHHHLPVV)₁₋₁₀;
(ADP1H)
(SEQ ID NO: 3)
(HTLQPHHHIPVV)₁₋₁₀;
(ADP2M)
(SEQ ID NO: 5)
(VPGHHSMTPTQH)₁₋₁₀;
(ADP2H)
(SEQ ID NO: 6)
(VPGQHSMTPIQH)₁₋₁₀;
(ADP4M)
(SEQ ID NO: 11)
(HPPSHTLQPHHELPVV)₁₋₁₀;
(ADP4H)
(SEQ ID NO: 12)
(HPPTHTLQPHHHIPVV)₁₋₁₀;
(ADP7M)
(SEQ ID NO: 19)
(HPPSHTLQPHHELPVVPAQQPVAPQQPMMPVPGHHSMTPTQH)₁₋₁₀;
(ADP7H)
(SEQ ID NO: 20)
(HPPTHTLQPHHHIPVVPAQQPVIPQQPMMPVPGQHSMTPIQH)₁₋₁₀;
(ADP8M)
(SEQ ID NO: 22)
(PAQQPVAPQQPMMP)₁₋₁₀;
and
(ADP8H)
(SEQ ID NO: 23)
(PAQQPVIPQQPMMP)₁₋₁₀;
or a functional equivalent thereof.
